(19) United States
(12) Patent Application Publication
(10) Pub. No.: US 2008/0200373 A1
(43) Pub. Date: Aug. 21, 2008

(54) Multi-Component Biological Transport Systems

(76) Inventors: Jacob M. Waugh, Mountain View, CA (US); Michael D. Dake, Stanford, CA (US)

Correspondence Address: KING & SPALDING, 1185 AVENUE OF THE AMERICAS, NEW YORK, NY, 10036-4003, US

(21) Appl. No.: 10/591,485

(22) PCT Filed: Mar. 3, 2005
(86) PCT No.: PCT/US05/06930

§ 371 Date: Nov. 7, 2007

(30) Foreign Application Priority Data
Mar. 3, 2004 (US) 10793138

(51) Int. Cl. A61K 38/16; A61K 38/02; A61P 21/02; A61Q 19/00

(52) U.S. Cl. 514/12; 514/2

(57) ABSTRACT

Compositions and methods are provided that are useful for the delivery, including transdermal delivery, of biologically active agents, such as non-protein non-nucleotide therapeutics and protein-based therapeutics excluding insulin, botulinum toxins, antibody fragments, and VEGF. The compositions and methods are particularly useful for topical delivery of antifungal agents and antigenic agents suitable for immunization. Alternately, the compositions can be prepared with components useful for targeting the delivery of the compositions as well as imaging components.

### CROSS-REFERENCES TO RELATED APPLICATIONS

This application is a continuation-in-part of U.S. application Ser. No. 09/910,432 filed Jul. 20, 2001, which in turn claims priority to U.S. Provisional Application Ser. No. 60/220,244, filed Jul. 21, 2000, the contents of which are incorporated herein by reference in its entirety.

### STATEMENT AS TO RIGHTS TO INVENTIONS MADE UNDER FEDERALLY SPONSORED RESEARCH AND DEVELOPMENT

Not applicable

### BACKGROUND OF THE INVENTION

Gene delivery systems can be broadly classified into two groups: viral and nonviral. Viral systems have major toxicity risks and have resulted in major complications and death in clinical trials. Nonviral systems are far less efficient than viral approaches but offer the potential to tailor applications to enhance specificity and potentially decrease toxicity. Nonviral strategies can be broadly classified as lipid-based or nonlipid-based. The strategy presented in this invention can he applied to any of the existing nonviral approaches, so all will be described here.

The simplest nonviral system is direct delivery of DNA. Due to the negative charge of DNA, very little of the DNA actually enters the cell and most is degraded. Virtually none of the DNA enters the nucleus without a nuclear targeting sequence in the strategy. Conventionally, another factor is employed to enhance the efficiency of gene/product delivery (DNA, RNA, or more recently protein therapeutics) either by mechanical effects such as electroporation, ultrasound, “gene gun” and direct microinjection, or by charge neutralization and chemical effects with agents such as calcium phosphate, polylysine, and liposome preparations. In the latter strategies, charge neutralization has been shown to increase nonspecific efficiencies several-fold over even chemical/mechanical effects of liposome preparations alone. Based upon these and similar results, many have concluded that DNA and RNA require charge neutralization for efficiency in cellular uptake, since DNA's negative charge essentially precludes transport except by endolysis with subsequent lysosome fusion (escaped with addition of other agents). Most transfection agents actually use an excess of positive charge in ratios of 2-4 fold over the net DNA negative charge. The resulting positive hybrid binds ionically to negatively-charged cell surface proteoglycans and dramatically enhances subsequent uptake. Some transfection agents seem to have a cellular tropism, most likely because of steric and charge patterns that more effectively target particular proteoglycans, which vary in cell-type specific patterns. Even with appropriate agents (i.e., correct tropism), charge neutralization alone or in combination with liposomes remains extremely inefficient relative to viral strategies. Thus, the community has identified a number of peptides and peptide fragments which facilitate efficient entry of a complex into a cell and past any endolysosome stage. Several such transport factors even allow efficient nuclear entry. In one process, the transport factor is directly linked to the therapeutic product of interest (small drug, gene, protein, etc). This approach requires that a new drug attached to the transport factor be produced, purified and tested. In many cases, these hybrids will actually constitute new drugs and will require full testing. Such a process results in significant additional risk and expense. Alternately, a number of strategies merely employ mixing of the agent nonspecifically (or even specifically at the surface) into liposome preparations as carriers for a drug/DNA/factor. Although an improvement over direct or simpler modalities in terms of efficiencies, these approaches remain inefficient (relative to virus) and considerably more toxic than simple nonviral strategies. Part of this inefficiency is due to poor nuclear translocation. As a result, strategies have evolved to add nuclear translocation signals to the complex detailed above, either as part of the therapeutic factor hybrid or as part of the liposome mixture. Additional refinements have included efforts to reduce DNA/RNA/factor degradation.

Perhaps the most important refinements in the basic strategies presented above have included specific ligands or other targeting agents together with the therapeutic factor. These strategies offer the potential for greatly reduced nonspecific toxicity and substantial improvements in efficiency, particularly when combined with efficiency agents described as above. However, the current strategies rely on covalent linkages to a single carrier and thus necessitate a specific synthesis (to assure that steric considerations in a degree of substitution scheme don't favor a single factor over the others—i.e., to assure that each efficiency factor and each imaging moiety, and each targeting moiety is present on the backbone). This renders virtually impossible a number of specific constructs (for example, sialyl-lewis X and an Fab fragment to a surface antigen, since steric limitations would prevent efficient binding of one or the other in most schemes, and in turn would interfere with efficiency factors). While promising in concept, these approaches represent expensive, very low yield (in terms of synthesis), and unproven solutions to this problem.

As must be evident, with each stage of development in nonviral gene and factor delivery, problems have been encountered and, in the next stage, solved with an added degree of complexity. Each improvement represented an incremental step over the prior standard. However, the added complexity brings risk from a patient-care standpoint and inefficiency and expense from a production standpoint. These barriers have led to greatly decreased enthusiasm for these otherwise promising potential therapies.

What is needed are new methods and compositions that are broadly applicable to compositions of diverse therapeutic or cosmeceutical agents that can be targeted or imaged to maximize delivery to a particular site. Surprisingly, the present invention provides such compositions and methods.

This invention further relates to formulations for transdermal delivery of proteins such as insulin, and also of larger therapeutic and diagnostic substances, for example, such substances having a molecular weight of 50,000 and higher including proteins such as botulinum toxin or other biologically active agents such as, for example, insulin, botulinum toxin, a therapeutic protein which does not therapeutically alter blood glucose levels, a nucleic acid-based agent, a non-protein non-nucleic acid therapeutic agent such as certain antifungals or alternately an agent for immunization. The invention specifically excludes antibody fragments which do not have biological activity other than only binding a specific antigen when the term “therapeutic” or “biologically active protein” is employed. Since antigens suitable for immunization have other biological activities such as mounting an immune response, these remain included in the appropriate aspects of this invention, however. Moreover, agents that have a biological activity or a therapeutic effect by binding a specific antigen, thereby blocking ligand binding or altering the conformation of the antigen are included in this invention.

Botulinum toxins (also known as botulin toxins or botulinum neurotoxins) are neurotoxins produced by the gram-positive bacteria Clostridium botulinum. They act to produce paralysis of muscles by preventing synoptic transmission or release of acetylcholine across the neuromuscular junction, and are thought to act in other ways as well. Their action essentially blocks signals that normally would cause muscle spasms or contractions, resulting in paralysis or would cause glandular secretions or overexcretion such as hyperhidrosis or acne.

Botulinum toxin is classified into eight neurotoxins that are serologically related, but distinct. Of these, seven can cause paralysis, namely botulinum neurotoxin serotypes A, B, C, D, E, F and G. Each of these is distinguished by neutralization with type-specific antibodies. Each type can be naturally-occurring, recombinant in production or engineered variants such as protein fusions. Nonetheless, the molecular weight of the botulinum toxin protein molecule, for all seven of these naturally-occurring active botulinum toxin serotypes or their recombinant forms, is about 150 kD. As released by the bacterium, the botulinum toxins are complexes comprising the 150 kD botulinum toxin protein molecule in question along with associated non-toxin proteins. The botulinum toxin type A complex can be produced by Clostridia bacterium as 900 kD, 500 kD and 300 kD forms. Botulinum toxin types B and C are apparently produced as only a 700 kD or 500 kD complex. Botulinum toxin type D is produced as both 300 kD and 500 kD complexes. Botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg, about 12 times the primate LD₅₀ for type A. Due to the molecule size and molecular structure of botulinum toxin, it cannot cross stratum corneum and the multiple layers of the underlying skin architecture.

Botulinum toxin type A is said to be the most lethal natural biological agent known to man. Spores of C. botulinum are found in soil and can grow in improperly sterilized and sealed food containers. Ingestion of the bacteria can cause botulism, which can be fatal. At the same time, the muscle-paralyzing effects of botulinum toxin have been used for therapeutic effects. Controlled administration of botulinum toxin has been used to provide muscle paralysis to treat conditions, for example, neuromuscular disorders characterized by hyperactive skeletal muscles. Conditions that have been treated with botulinum toxin include hemifacial spasm, adult onset spasmodic torticollis, anal fissure, blepharospasm, cerebral palsy, cervical dystonia, migraine headaches, strabismus, temperomandibular joint disorder, and various types of muscle cramping and spasms. More recently the muscle-paralyzing effects of botulinum toxin have been taken advantage of in therapeutic and cosmetic facial applications such as treatment of wrinkles, frown lines, and other results of spasms or contractions of facial muscles.

Botulism, the characteristic symptom complex from systemic botulinum toxin exposure, has existed in Europe since antiquity. In 1895, Emile P. van Ermengem first isolated the anaerobic spore-forming bacillus from raw salted pork meat obtained from post-mortem tissue of victims who died of botulism in Belgium. Van Ermengem found the disease to be caused by an extracellular toxin that was produced by what he called Bacillus botulinus (Van Ermengem, Z Hyyg Infektionskr, 26:1-56; Rev Infect (1897)). The name was changed in 1922 to Clostridium botulinum. The name Clostridium was used to reflect the anaerobic nature of the microorganism and also its morphologic characteristics (Carruthers and Carruthers, Can J Ophthalmol, 31:389-400 (1996)). In the 1920's, a crude form of Botulinum toxin type A was isolated after additional outbreaks of food poisoning. Dr. Herman Sommer at the University of California, San Francisco made the first attempts to purify the neurotoxin (Borodic et al., Ophthalmic Plast Recostr Surg, 7:54-60 (1991)). In 1946, Dr. Edward J. Schantz and his colleagues isolated the neurotoxin in crystalline form (Schantz et al., In: Jankovi J, Hallet M (Eds) Therapy with Botulinum Toxin, New York, N.Y.: Marcel Dekker, 41-49 (1994)). By 1949, Burgen and his associates were able to demonstrate that the Botulinum toxin blocks impulses across the neuromuscular junction (Burgen et al., J Physiol, 109:10-24 (1949)). Allan B. Scott first used botulinum toxin A (BTX-A) in monkeys in 1973. Scott demonstrated reversible ocular muscle paralysis lasting 3 months (Lamanna, Science, 130:763-772 (1959)). Soon afterwards, BTX-A was reported to be a successful treatment in humans for strabismus, blepharospasm, and spasmodic torticollis (Baron et al., In: Baron E J, Peterson L R, Finegold S M (Eds), Bailey & Scotts Diagnostic Microbiology, St. Louis, Mo.: Mosby Year Book, 504-523 (1994); Carruthers and Carruthers, Adv Dermatol, 12:325-348 (1997); Markowitz, In: Strickland G T (Eds) Hunters Tropical Medicine, 7^{th} ed. Philadelphia: W. B. Saunders, 441-444 (1991)). In 1986, Jean and Alastair Carruthers, a husband and wife team consisting of an ocuplastic surgeon and a dermatologist, began to evolve the cosmetic use of botulinum toxin-A (BTX-A) for treatment of movement-associated wrinkles in the glabella area (Schantz and Scott, In Lewis GE (Ed) Biomedical Aspects of Botulinum, New York: Academic Press, 143-150 (1981)). The Carruthers' use of BTX-A for the treatment of wrinkles led to their seminal publication of this approach in 1992 (Schantz and Scott, In Lewis GE (Ed) Biomedical Aspects of Botulinum, New York: Academic Press, 143-150 (1981)). By 1994, the same team reported experiences with other movement-associated wrinkles on the face (Scott, Ophthalmol, 87:1044-1049 (1980)). This in turn led to the birth of the era of cosmetic BTX-A treatment.

Skin protects the body's organs from external environmental threats and acts as a thermostat to maintain body temperature. It consists of several different layers, each with specialized functions. The major layers include the epidermis, the dermis and the hypodermis. The epidermis is a stratifying layer of epithelial cells that overlies the dermis, which consists of connective tissue. Both the epidermis and the dermis are further supported by the hypodermis, an internal layer of adipose tissue.

The epidermis, the topmost layer of skin, is only 0.1 to 1.5 millimeters thick (Inlander, Skin, New York, N.Y.: People's Medical Society, 1-7 (1998)). It consists of keratinocytes and is divided into several layers based on their state of differentiation. The epidermis can be further classified into the stratum corneum and the viable epidermis, which consists of the granular melphigian and basal cells. The stratum corneum is hygroscopic and requires at least 10% moisture by weight to maintain its flexibility and softness. The hygroscopicity is attributable in part to the water-holding capacity of keratin. When the horny layer loses its softness and flexibility it becomes rough and brittle, resulting in dry skin.

The dermis, which lies just beneath the epidermis, is 1.5 to 4 millimeters thick It is the thickest of the three layers of the skin. In addition, the dermis is also home to most of the skin's structures, including sweat and oil glands (which secrete substances through openings in the skin called pores, or comedos), hair follicles, nerve endings, and blood and lymph vessels (Inlander, Skin, New York, N.Y.: People's Medical Society, 1-7 (1998)). However, the main components of the dermis are collagen and elastin.

The hypodermis is the deepest layer of the skin. It acts both as an insulator for body heat conservation and as a shock absorber for organ protection (Inlander, Skin, New York, N.Y.: People's Medical Society, 1-7 (1998)). In addition, the hypodermis also stores fat for energy reserves. The pH of skin is normally between 5 and 6. This acidity is due to the presence of amphoteric amino acids, lactic acid, and fatty acids from the secretions of the sebaceous glands. The term “acid mantle” refers to the presence of the water-soluble substances on most regions of the skin. The buffering capacity of the skin is due in part to these secretions stored in the skin's horny layer.

Wrinkles, one of the telltale signs of aging, can be caused by biochemical, histological, and physiologic changes that accumulate from environmental damage (Benedetto, International Journal of Dermatology, 38:641-655 (1999)). In addition, there are other secondary factors that can cause characteristic folds, furrows, and creases of facial wrinkles (Stegman et al., The Skin of the Aging Face Cosmetic Dermatological Surgery, 2^{nd} ed., St. Louis, Mo.: Mosby Year Book: 5-15 (1990)). These secondary factors include the constant pull of gravity, frequent and constant positional pressure on the skin (i.e., during sleep), and repeated facial movements caused by the contraction of facial muscles (Stegman et al., The Skin of the Aging Face Cosmetic Dermatological Surgery, 2^{nd} ed., St. Louis, Mo.: Mosby Year Book: 5-15 (1990)). Different techniques have been utilized in order potentially to mollify some of the signs of aging. These techniques range from facial moisturizers containing alpha hydroxy acids and retinol to surgical procedures and injections of neurotoxins.

One of the principal functions of skin is to provide a barrier to the transportation of water and substances potentially harmful to normal homeostasis. The body would rapidly dehydrate without a tough, semi-permeable skin. The skin helps to prevent the entry of harmful substances into the body. Although most substances cannot penetrate the barrier, a number of strategies have been developed to selectively increase the permeability of skin with variable success.

Since BTX cannot penetrate the skin efficiently, in order to provide the therapeutic effects of BTX the toxin must currently be injected into the skin. The Federal Food and Drug Administration has approved such a procedure, for treatment of wrinkles, and BTX products are now marketed for this treatment. In such treatments, the botulinum toxin is administered by carefully controlled or monitored injection, creating large wells of toxin at the treatment site. However, such treatment can be uncomfortable and more typically involves some pain.

Topical application of botulinum toxin provides for a safer and more desirable treatment alternative due to painless nature of application, the larger treatment surface area that can be covered, the ability to formulate a pure toxin with higher specific activity, reduced training to apply the botulinum therapeutic, smaller doses necessary to effect, and large wells of toxin are not required in order to reach a therapeutic clinical result.

Transdermal administration of other therapeutics is also an area of great interest due, for instance, to the potential for decreased patient discomfort, direct administration of therapeutic agents into the bloodstream, and the opportunities for monitored delivery via the use of specially constructed devices and/or of controlled release formulations and techniques. One substance for which ease of administration is desired is insulin, which in many cases must still be administered by injection (including self-injection). Ease of administration would also be advantageous for larger proteins such as botulinum toxin. Other agents which do not readily cross skin but are substantially smaller than insulin or have different physiochemical properties and thus very different rates and abilities to cross skin with or without additional materials to facilitate this transfer. Further interaction of each with materials to facilitate transfer is unique for each.

### SUMMARY OF THE INVENTION

In one aspect, the present invention provides a composition comprising a non-covalent complex of:
a) a positively-charged backbone; and
b) at least two members selected from the group consisting of:
   i) a first negatively-charged backbone having a plurality of attached imaging moieties; or alternatively a plurality of negatively-charged imaging moieties;
   ii) a second negatively-charged backbone having a plurality of attached targeting agents, or alternatively a plurality of negatively-charged targeting moieties;
   iii) at least one member selected from RNA, DNA, ribozymes, modified oligonucleic acids and cDNA encoding a selected transgene;
   iv) DNA encoding at least one persistence factor; and
   v) a third negatively-charged backbone having a plurality of attached biological agents, or a negatively-charged biological agent;
wherein the complex carries a net positive charge and at least one of the members is selected from i), ii), iii) or v).

The biological agent, in this aspect of the invention, can be either a therapeutic agent or a cosmeceutical agent. The invention specifically excludes antibody fragments which do not have biological activity other than only binding a specific antigen when the term “therapeutic” or “biologically active protein” is employed. Since antigens suitable for immunization have other biological activities such as mounting an immune response, these remain included in the appropriate aspects of this invention, however. Moreover, agents that have a biological activity or a therapeutic effect by binding a specific antigen, thereby blocking ligand binding or altering the conformation of the antigen are included in this invention. Alternatively, candidate agents can be used to determine in vivo efficacy in these non-covalent complexes.

In another aspect, the present invention provides a composition comprising a non-covalent complex of a positively-charged backbone having at least one attached efficiency group and at least one nucleic acid member selected from the group consisting of RNA, DNA, ribozymes, modified oligonucleic acids and cDNA encoding a selected transgene.

In another aspect, the present invention provides a method for delivery of a biological agent to a cell surface in a subject, said method comprising administering to said subject a composition as described above.

In yet another aspect, the present invention provides a method for preparing a pharmaceutical or cosmeceutical composition, the method comprising combining a positively charged backbone component and at least two members selected from the group consisting of:
i) a first negatively-charged backbone having a plurality of attached imaging moieties, or alternatively a plurality of negatively-charged imaging moieties;
ii) a second negatively-charged backbone having a plurality of attached targeting agents, or alternatively a plurality of negatively-charged targeting moieties;
iii) at least one member selected from RNA, DNA, ribozymes, modified oligonucleic acids and cDNA encoding a selected transgene;
iv) DNA encoding at least one persistence factor; and
v) a third negatively-charged backbone having a plurality of attached biological agents or cosmeceutical agents, or a negatively-charged biological agent or cosmeceutical agent;
   with a pharmaceutically or cosmeceutically acceptable carrier to form a non-covalent complex having a net positive charge, with the proviso that at least one of said members is selected from i), ii), iii) or v).

In still another aspect, the present invention provides a kit for formulating a pharmaceutical or cosmeceutical delivery composition, the kit comprising a positively charged backbone component and at least two components selected from groups i) through v) above, along with instructions for preparing the delivery composition.

In yet another aspect, this invention relates to a composition comprising a biologically active agent such as insulin, botulinum toxin, other proteins which do not therapeutically alter blood glucose levels, a nucleic acid-based agent, a non-protein non-nucleic acid therapeutic agent such as certain antifungals or alternately an agent for immunization, and a carrier comprising a positively charged carrier having a backbone with attached positively charged branching or “efficiency” groups, all as described herein. The invention specifically excludes antibody fragments which do not have biological activity other than only binding a specific antigen when the term “therapeutic” or “biologically active protein” is employed. Since antigens suitable for immunization have other biological activities such as mounting an immune response, these remain included in the appropriate aspects of this invention, however. Moreover, agents that have a biological activity or a therapeutic effect by binding a specific antigen, thereby blocking ligand binding or altering the conformation of the antigen are included in this invention. The biologically active agent is preferably insulin, botulinum toxin (BTX), an antigen for immunization, or certain antifungal agents. Suitable antifungal agents include, for example, amphotericin B, fluconazole, flucytosine, itraconazole, ketoconazole, clotrimazole, econozole, griseofulvin, miconazole, nystatin, ciclopirox and the like. Most preferably the positively charged carrier is a comparatively short- or medium-chain positively charged polypeptide or a positively charged nonpeptidyl polymer, for example, a polyalkyleneimine. When the biologically active agent is botulinum toxin, the invention further relates to a method for producing a biologic effect such as muscle paralysis, reducing hypersecretion or sweating, treating neurologic pain or migraine headache, reducing muscle spasms, preventing or reducing acne, or reducing or enhancing an immune response, by topically applying a composition containing an effective amount of botulinum toxin, preferably to the skin, of a subject or patient in need of such treatment. The invention also relates to a method for producing an aesthetic and/or cosmetic effect, for example by topical application of botulinum toxin to the face instead of by injection into facial muscles. When the biologically active agent is insulin, the invention relates to a method of transdermally delivering insulin to a subject by applying to the skin or epithelium of the subject an effective amount of such a composition containing insulin, or a combination of insulin and the positively charged backbone. Proteins that are not normally capable of crossing the skin or epithelium appreciably relative to the complex of the same agent and the carriers of the present invention and that do not have a therapeutic effect on lowering blood glucose have widely differing surface and physiochemical properties from insulin that normally would make it uncertain whether a technique that afforded transdermal delivery of insulin would have positive results for any other proteins. However, carriers of this invention that have a positively charged backbone with positively charged branching groups, as described herein are quite surprisingly capable of providing transdermal delivery of such other proteins, including, for example botulinum toxin. Particular carriers suited for transdermal delivery of particular proteins can easily be identified using tests such as those described in the Examples. Such a protein may, for example be a large protein having a molecular weight over 50,000 kD or under 20,000 kD. As used herein, the word “therapeutic” in the context of blood glucose refers to a decline in blood glucose levels sufficient to alleviate acute symptoms or signs of hyperglycemia, for example in diabetic patients. In all aspects of the present invention, the association between the carrier and the biologically active agent is by non-covalent interaction, which can include, for example, ionic interactions, hydrogen bonding, van der Waals forces, or combinations thereof. In certain aspects of the invention, transdermal delivery of therapeutic proteins capable of achieving therapeutic alterations of blood glucose are specifically excluded. As employed herein, the antigenic agents suitable for immunization can be protein-based antigens which do not therapeutically alter blood glucose levels, non-protein non-nucleic acid agents or hybrids thereof. Nucleic acids encoding antigens are specifically not suitable for the compositions of the present invention, however. Thus, the agents included are themselves antigens suitable for immunization. Suitable antigens include, for example, those for environmental agents, pathogens or biohazards. Suitable agents preferably include, for example, antigens related to botulism, malaria, rabies, anthrax, tuberculosis, or related to childhood immunizations such as hepatitis B, diphtheria, pertussis, tetanus, Haemophilus influenza type b, inactivated poliovirus, measles, mumps, rubella, varicella, pneumococcus, hepatitis A, and influenza.

The positively charged carriers or backbones with their positively charged branching groups, as described herein, are themselves novel compounds, and form another aspect of this invention.

This invention also provides a method for preparing a pharmaceutical or cosmeceutical composition that comprises combining a carrier comprising a positively charged polypeptide or a positively charged nonpeptidyl polymer such as a long-chain polyalkyleneimine, the polypeptide or nonpeptidyl polymer having positively charged branching or “efficiency” groups as defined herein, with a biologically active agent such as, for example, insulin, botulinum toxin, a therapeutic protein which does not therapeutically alter blood glucose levels, a nucleic acid-based agent, a non-protein non-nucleic acid therapeutic agent such as certain antifungals or alternately an agent for immunization. The invention also provides a kit for preparing or formulating such a composition that comprises the carrier and the therapeutic substance, as well as such additional items that are needed to produce a usable formulation, or a premix that may in turn be used to produce such a formulation. Such a kit may consist of an applicator or other device for applications of the compositions or components thereof and methods of the present invention. As used herein, “device” can refer for example to an instrument or applicator for delivery or for mixing or other preparation technique to form or apply the compositions and methods of the present invention.

This invention also comprises devices for transdermal transmission of a biologically active agent such as, for example, insulin, botulinum toxin, a therapeutic protein which does not therapeutically alter blood glucose levels, a nucleic acid-based agent, a non-protein non-nucleic acid therapeutic agent such as certain antifungals or alternately an agent for immunization that is contained within a composition that, in turn, in one embodiment, comprises a carrier comprising a positively charged polypeptide of preferably short chain to intermediate chain length or a longer-chain nonpeptidyl polymeric carrier that has positively charged branching or “efficiency” groups as defined herein, and a therapeutic agent as just mentioned. Such devices may be as simple in construction as a skin patch, or may be a more complicated device that includes means for dispensing and monitoring the dispensing of the composition, and optionally means for monitoring the condition of the subject in one or more aspects, including monitoring the reaction of the subject to the substances being dispensed. In all aspects of the present invention, the association between the carrier and the biologically active agent is by non-covalent interaction, which can include, for example, ionic interactions, hydrogen bonding, van der Waals forces, or combinations thereof.

Alternatively the device may contain only the therapeutic biologically active agent for example, insulin, botulinum toxin, a therapeutic protein which does not therapeutically alter blood glucose levels, a nucleic acid-based agent, a non-protein non-nucleic acid therapeutic agent such as certain antifungals or alternately an agent for immunization, and the carrier may be applied separately to the skin. Accordingly, the invention also comprises a kit that includes both a device for dispensing via the skin and a material that contains the positively charged carrier or backbone, and that is suitable for applying to the skin or epithelium of a subject.

In general, the invention also comprises a method for administering a biologically active agent such as, for example, insulin, botulinum toxin, a therapeutic protein which does not therapeutically alter blood glucose levels, a nucleic acid-based agent, a non-protein non-nucleic acid therapeutic agent such as certain antifungals or alternately an agent for immunization to a subject or patient in need thereof, comprising topically administering an effective amount of said biologically active agent in conjunction with a positively charged polypeptide or non-polypeptidyl polymer such as a polyalkyleneimine having positively charged branching groups, as described herein. By “in conjunction with” is meant that the two components—biologically active agent and positively charged carrier—are administered in a combination procedure, which may involve either combining them in a composition, which is then administered to the subject, or administering them separately, but in a manner such that they act together to provide the requisite delivery of an effective amount of the biologically active agent. For example, a composition containing the positively charged carrier may first be applied to the skin of the subject, followed by applying a skin patch or other device containing the biologically active agent.

The invention also relates to methods of applying biologically active agents such as, for example, insulin, botulinum toxin, a therapeutic protein which does not therapeutically alter blood glucose levels, a nucleic acid-based agent, a non-protein non-nucleic acid therapeutic agent such as certain antifungals or alternately an agent for immunization as defined herein to epithelial cells, including those other than epithelial skin cells, for example, epithelia ophthalmic cells or cells of the gastrointestinal system.

### BRIEF DESCRIPTION OF THE DRAWINGS

FIG. 1 provides a schematic representation the components used in the invention.

FIG. 2 provides a schematic representation of several embodiments of the invention.

FIGS. 3-4 represent the results of transdermal delivery of a plasmid containing the transgene for E. coli beta-galactosidase as described in Example 2.

FIG. 5 represents the results of transdermal delivery of a plasmid containing the transgene for E. coli beta-galactosidase as described in Example 3.

FIG. 6 represents the results of transdermal delivery of a plasmid containing the transgene for E. coli beta-galactosidase as described in Example 4.

FIG. 7 represents the results of transdermal delivery of a botulinum toxin as described in Example 5.

FIG. 8 is a photographic depiction of the results of transdermal delivery of a botulinum toxin as described in Example 6.

FIG. 9 is a photographic depiction that the imaging complexes of Example 9 follow the brightfield distribution (panels a and c) for melanoma pigmented cells with fluorescent optical imaging agents (panels b and d) for two different fields and different magnifications (panels a and b at 10× versus panels c and d at 40× magnifications).

### DESCRIPTION OF THE INVENTION

### General

The present invention provides a component-based system for selective, persistent, delivery of imaging agents, genes or other therapeutic agents. Individual features for the compositions can be selected by designating desired components in bedside formulations. Additionally, in one aspect imaging and specific targeting moieties are provided on separate negatively charged backbones which will form a non-covalent ionic complex with a positive backbone. By placing these components on a negatively charged backbone, the invention obviates the need for attaching components in precise locations on a positive backbone as employed in other strategies (increasing complexity and expense and decreasing efficiency to a level that no successful combination has yet been reported due to steric limitations).

In another aspect, certain substances can be transdermally delivered by use of certain positively charged carriers alone, without requiring the inclusion of a negative backbone. In these cases, the substance or a derivative thereof have sufficient negative charge to associate with the positively charged carriers of the present invention non-covalently. The term “sufficient” in this context refers to an association that can be determined for example by change in particle sizing or functional spectrophotometry versus the components alone.

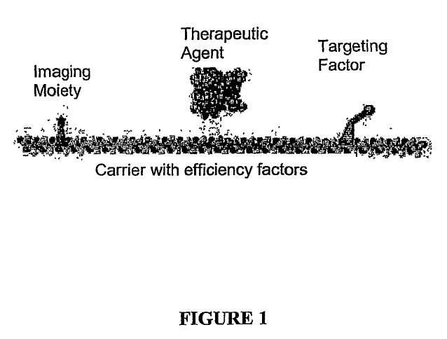

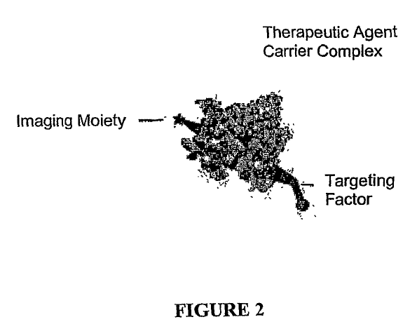

Further understanding of the invention is provided with reference to FIG. 1. In this figure, the components are shown as (1) a solid backbone having attached positively charged groups (also referred to as efficiency groups shown as darkened circles attached to a darkened bar), for example (Gly)ₙ₁-(Arg)ₙ₂ (wherein the subscript n1 is an integer of from 3 to about 5, and the subscript n2 is an odd integer of from about 7 to about 17) or TAT domains; (2) a short negatively charged backbone having attached imaging moieties (open triangles attached to a light bar); (3) a short negatively charged backbone having attached targeting agents and/or therapeutic agents (open circles attached to a light bar); (4) an oligonucleic acid, RNA, DNA or cDNA (light cross hatched bar); and (5) DNA encoding persistence factors (dark cross hatched bar). FIG. 2 illustrates various examples of multicomponent compositions wherein the groups are depicted as set out in FIG. 1. For example, in FIG. 2, a first multi-component composition is illustrated in which a positively charged backbone has associated an imaging component, a targeting component, an oligonucleic acid and a persistence factor.

A second multi-component composition is illustrated which is designed for diagnostic/prognostic imaging. In this composition the positively charged backbone is complexed with both imaging components and targeting components. Finally, a third multi-component system is illustrated which is useful for gene delivery. In this system, a complex is formed between a positively charged backbone, a targeting component, a gene of interest and DNA encoding a persistence factor. The present invention, described more fully below, provides a number of additional compositions useful in therapeutic and diagnostic programs.

### Description of the Embodiments

Compositions

In view of the above, the present invention provides in one aspect a composition comprising a non-covalent complex of:
a) a positively-charged backbone; and
b) at least two members selected from the group consisting of:
   i) a first negatively-charged backbone having a plurality of attached imaging moieties; or alternatively a plurality of negatively-charged imaging moieties;
   ii) a second negatively-charged backbone having a plurality of attached targeting agents; or alternatively a plurality of negatively-charged targeting moieties;
   iii) at least one member selected from RNA, DNA, ribozymes, modified oligonucleic acids and cDNA encoding a selected transgene;
   iv) DNA encoding at least one persistence factor; and
   v) a third negatively-charged backbone having a plurality of attached biological agents, or a negatively-charged biological agent;
wherein the complex carries a net positive charge and at least one of the members is selected from i), ii) iii) or v).

In one group of embodiments, the composition comprises at least three members selected from groups i) through v). In another group of embodiments, the composition comprises at least one member from each of groups i), ii), iii) and iv). In yet another group of embodiments, the composition comprises at least one member from each of groups i) and ii). In another group of embodiments, the composition comprises at least one member from each of groups ii), iii) and iv).

Preferably, the positively-charged backbone has a length of from about 1 to 4 times the combined lengths of the members from group b). Alternatively, the positively charged backbone has a charge ratio of from about 1 to 4 times the combined charge of the members from group b). In some embodiments, the charge density is uniform and the length and charge ratios are approximately the same. Size to size (length) ratios can be determined based on molecular studies of the components or can be determined from the masses of the components

By “positively charged” is meant that the carrier has a positive charge under at least some solution-phase conditions, more preferably at least under some physiologically compatible conditions. More specifically, “positively charged” as used herein, means that the group in question contains functionalities that are charged under all pH conditions, such as a quaternary amine, or containing a functionality which can acquire positive charge under certain solution-phase conditions, such as pH changes in the case of primary amines. More preferably, “positively charged” as used herein refers to those that have the behavior of associating with anions over physiologically compatible conditions. Polymers with a multiplicity of positively-charged moieties need not be homopolymers, as will be apparent to one skilled in the art. Other examples of positively charged moieties are well known in the prior art and can be employed readily, as will be apparent to those skilled in the art. The positively charged carriers described in this invention which themselves do not have a therapeutic activity are novel compounds which have utility for example in compositions and methods as described herein. Thus, in another aspect of the present invention, we detail these novel compounds which include any carrier which comprises a positively charged backbone having attached positively charged branching groups as described herein and which does not itself have a therapeutic biologic activity. The invention specifically excludes antibody fragments which do not have biological activity other than only binding a specific antigen when the term “therapeutic” or “biologically active protein” is employed. Since antigens suitable for immunization have other biological activities such as mounting an immune response, these remain included in the appropriate aspects of this invention, however. Moreover, agents that have a biological activity or a therapeutic effect by binding a specific antigen, thereby blocking ligand binding or altering the conformation of the antigen are included in this invention.

In another embodiment, the present invention provides in one aspect a composition comprising a biologically active agent such as, for example, insulin, botulinum toxin, a therapeutic protein which does not therapeutically alter blood glucose levels, a nucleic acid-based agent, a non-protein non-nucleic acid therapeutic agent such as certain antifungals or alternately an agent for immunization and a carrier comprising a positively charged backbone, for instance a positively charged polypeptide or nonpeptidyl polymer, which may be either a hetero- or homopolymer, such as a polyalkyleneimine, the polypeptide or nonpeptidyl polymer having positively charged branching or “efficiency” groups as defined herein. Each protein-based therapeutic and non-nucleic acid non-protein therapeutic has distinct physiochemical properties which alter total complex characteristics. Such positively charged carriers are among the materials described below as positively charged backbones. The invention also provides a method for administering a therapeutically effective amount of a biologically active agent as mentioned herein, comprising applying to the skin or epithelium of the subject (which may be a human or other mammal) the biologically active agent and an amount of the positively charged backbone having branching groups that is effective to provide transdermal delivery of the biologically active agent to the subject. In that method, the biologically active agent and the positively charged carrier may be applied as a pre-mixed composition, or may be applied separately to the skin or epithelium (for instance, the agent may be in a skin patch or other device and the carrier may be contained in a liquid or other type of composition that is applied to the skin before application of the skin patch). As used herein, the word “therapeutic” in the context of blood glucose refers to a decline in blood glucose levels sufficient to alleviate acute symptoms or signs of hyperglycemia, for example in diabetic patients. In certain aspects of the invention, transdermal delivery of therapeutic proteins capable of achieving therapeutic alterations of blood glucose is specifically excluded. The invention specifically excludes antibody fragments which do not have biological activity other than only binding a specific antigen when the term “therapeutic” or “biologically active protein” is employed. Since antigens suitable for immunization have other biological activities such as mounting an immune response, these remain included in the appropriate aspects of this invention, however. Moreover, agents that have a biological activity or a therapeutic effect by binding a specific antigen, thereby blocking ligand binding or altering the conformation of the antigen are included in this invention. As employed herein, the antigenic agents suitable for immunization can be protein-based antigens which do not therapeutically alter blood glucose levels, non-protein non-nucleic acid agents or hybrids thereof. Nucleic acids encoding antigens are specifically not suitable for the compositions of the present invention, however. Thus, the agents included are themselves antigens suitable for immunization. Suitable antigens include, for example, those for environmental agents, pathogens or biohazards. Suitable agents preferably include, for example, antigens related to botulism, malaria, rabies, anthrax, tuberculosis, or related to childhood immunizations such as hepatitis B, diphtheria, pertussis, tetanus, Haemophilus influenza type b, inactivated poliovirus, measles, mumps, rubella, varicella, pneumococcus, hepatitis A, and influenza.

Positively Charged Backbone

The positively-charged backbone (also referred to as a positively charged “carrier”) is typically a linear chain of atoms, either with groups in the chain carrying a positive charge at physiological pH, or with groups carrying a positive charge attached to side chains extending from the backbone. Preferably, the positively charged backbone itself will not have a defined enzymatic or biologic activity. The linear backbone is a hydrocarbon backbone which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like). In one group of embodiments, the positively charged backbone is a polypropyleneamine wherein a number of the amine nitrogen atoms are present as ammonium groups (tetra-substituted) carrying a positive charge. In another embodiment, the positively charged backbone is a nonpeptidyl polymer, which may be a hetero or homo-polymer, such as a polyalkyleneimine, for example a polyethyleneimine or polypropyleneimine, having a molecular weight of from about 10,000 to about 2,500,000, preferably from about 100,000 to about 1,800,000, and most preferably from about 500,000 to about 1,400,000. In another group of embodiments, the backbone has attached a plurality of side-chain moieties that include positively charged groups (e.g., ammonium groups, pyridinium groups, phosphonium groups, sulfonium groups, guanidinium groups, or amidinium groups). The sidechain moieties in this group of embodiments can be placed at spacings along the backbone that are consistent in separations or variable. Additionally, the length of the sidechains can be similar or dissimilar. For example, in one group of embodiments, the sidechains can be linear or branched hydrocarbon chains having from one to twenty carbon atoms and terminating at the distal end (away from the backbone) in one of the above-noted positively charged groups. In all aspects of the present invention, the association between the carrier and the biologically active agent is by non-covalent interaction, which can include, for example, ionic interactions, hydrogen bonding, van der Waals forces, or combinations thereof.

In one group of embodiments, the positively charged backbone is a polypeptide having multiple positively charged sidechain groups (e.g., lysine, arginine, ornithine, homoarginine, and the like). Preferably, the polypeptide has a molecular weight of from about 10,000 to about 1,500,000, more preferably from about 25,000 to about 1,200,000, most preferably from about 100,000 to about 1,000,000. One of skill in the art will appreciate that when amino acids are used in this portion of the invention, the sidechains can have either the D- or L-form (R or S configuration) at the center of attachment.

Alternatively, the backbone can be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the α-carbon atoms. As above, a portion of the sidechains will typically terminate in a positively charged group to provide a positively charged backbone component. Synthesis of peptoids is described in, for example, U.S. Pat. No. 5,877,278. As the term is used herein, positively charged backbones that have a peptoid backbone construction are considered “non-peptide” as they are not composed of amino acids having naturally occurring sidechains at the α-carbon locations.

A variety of other backbones can be used employing, for example, steric or electronic mimics of polypeptides wherein the amide linkages of the peptide are replaced with surrogates such as ester linkages, thioamides (—CSNH—), reversed thioamides (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from α-amino acids.

In each of the backbones provided above, sidechain groups can be appended that carry a positively charged group. For example, the sulfonamide-linked backbones (—SO₂NH— and —NHSO₂—) can have sidechain groups attached to the nitrogen atoms. Similarly, the hydroxyethylene (—CH(OH)CH₂—) linkage can bear a sidechain group attached to the hydroxy substituent. One of skill in the art can readily adapt the other linkage chemistries to provide positively charged sidechain groups using standard synthetic methods.

In a particularly preferred embodiment, the positively charged backbone is a polypeptide having branching groups (also referred to as efficiency groups) independently selected from -(gly)ₙ₁-(arg)ₙ₂, HIV-TAT or fragments thereof, or the protein transduction domain of Antennapedia, or a fragment or mixture thereof, in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. Still further preferred are those embodiments in which the HIV-TAT fragment has the formula (gly)ₚ-RGRDDRRQRRR-(gly)_{q}, (gly)ₚ-YGRKKRRQRRR-(gly)_{q} or (gly)ₚ-RKKRRQRRR-(gly)_{q} wherein the subscripts p and q are each independently an integer of from 0 to 20 and the fragment is attached to the backbone via either the C-terminus or the N-terminus of the fragment. Preferred HIV-TAT fragments are those in which the subscripts p and q are each independently integers of from 0 to 8, more preferably 2 to 5. In another preferred embodiment the positively charged side chain or branching group is the Antennapedia (Antp) protein transduction domain (PTD), or a fragment thereof that retains activity. Preferably the positively charged carrier includes side-chain positively charged branching groups in an amount of at least about 0.05%, as a percentage of the total carrier weight, preferably from about 0.05 to about 45 weight %, and most preferably from about 0.1 to about 30 weight %. For positively charged branching groups having the formula -(gly)ₙ₁-(arg)ₙ₂, the most preferred amount is from about 0.1 to about 25%.

In another particularly preferred embodiment, the backbone portion is a polylysine and positively charged branching groups are attached to the lysine sidechain amino groups. The polylysine used in this particularly preferred embodiment has a molecular weight of from about 10,000 to about 1,500,000, preferably from about 25,000 to about 1,200,000, and most preferably from about 100,000 to about 1,000,000. It can be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000. The appropriate selection of a polylysine will depend on the remaining components of the composition and will be sufficient to provide an overall net positive charge to the composition and provide a length that is preferably from one to four times the combined length of the negatively charged components. Preferred positively charged branching groups or efficiency groups include, for example, -gly-gly-gly-arg-arg-arg-arg-arg-arg-arg (-Gly₃Arg₇) or HIV-TAT. In another preferred embodiment the positively charged backbone is a long chain polyalkyleneimine such as a polyethyleneimine, for example, one having a molecular weight of about 1,000,000.

The positively charged backbones or carrier molecules comprising polypeptides or nonpeptidyl polymers such as polyalkyleneimines and other positively charged backbones mentioned above, having the branching groups described above, are novel compounds and form an aspect of this invention.

In one embodiment of the invention, only a positively charged carrier that has positively charged branching groups is necessary for transdermal delivery of the active substance. In one embodiment of this case the positively charged carrier is a polypeptide (e.g., lysine, arginine, ornithine, homoarginine, and the like) having multiple positively charged side-chain groups, as described above. Preferably, the polypeptide has a molecular weight of at least about 10,000. In another embodiment of this case the positively charged carrier is a nonpeptidyl polymer such as a polyalkyleneimine having multiple positively charged side-chain groups having a molecular weight of at least about 100,000. Such polyalkyleneimines include polyethylene- and polypropyleneimines. In either instance, for use as the sole necessary agent for transdermal delivery the positively charged carrier molecule includes positively charged branching or efficiency groups, comprising -(gly)ₙ₁-(arg)ₙ₂, in which the subscript n1 is an integer of from 0 to 20 more preferably 0 to 8, still more preferably 2 to 5, and the subscript n2 is independently an odd integer of from about 5 to about 25, more preferably from about 7 to about 17, and most preferably from about 7 to about 13, HIV-TAT or fragments thereof, or Antennapedia PTD or a fragment thereof. Preferably the side-chain or branching groups have the general formula -(gly)ₙ₁-(arg)ₙ₂ as described above. Other preferred embodiments are those in which the branching or efficiency groups are HIV-TAT fragments that have the formula (gly)ₚ-RGRDDRRQRRR-(gly), (gly)ₚ-YGRKKRRQRRR-(gly)_{q}, or (gly)ₚ-RKKRRQRRR-(gly)_{q}, wherein the subscripts p and q are each independently an integer of from 0 to 20 and the fragment is attached to the carrier molecule via either the C-terminus or the N-terminus of the fragment. The side branching groups can have either the D- or L-form (R or S configuration) at the center of attachment. Preferred HIV-TAT fragments are those in which the subscripts p and q are each independently integers of from 0 to 8, more preferably 2 to 5. Other preferred embodiments are those in which the branching groups are Antennapedia PTD groups or fragments thereof that retain the group's activity. These are known in the art, for instance, from Console et al., J. Biol. Chem. 278:35109 (2003).

In a particularly preferred embodiment, the carrier is a polylysine with positively charged branching groups attached to the lysine side-chain amino groups. The polylysine used in this particularly preferred embodiment can be any of the commercially available (Sigma Chemical Company, St. Louis, Mo., USA, e.g.) polylysines such as, for example, polylysine having MW>70,000, polylysine having MW of 70,000 to 150,000, polylysine having MW 150,000 to 300,000 and polylysine having MW>300,000. However, preferably the polylysine has MW of at least about 10,000. Preferred positively charged branching groups or efficiency groups include, for example, -gly-gly-gly-arg-arg-arg-arg-arg-arg-arg (-Gly₃Arg₇), HIV-TAT or fragments of it, and Antennapedia PTD or fragments thereof.

Other Components

In addition to the positively charged backbone component, the multicomponent compositions of the present invention comprise at least two components from the group consisting of the following:
i) a first negatively-charged backbone having a plurality of attached imaging moieties; or alternatively a plurality of negatively-charged imaging moieties;
ii) a second negatively-charged backbone having a plurality of attached targeting agents; or alternatively a plurality of negatively-charged targeting moieties;
iii) at least one member selected from RNA, DNA, ribozymes, modified oligonucleic acids and cDNA encoding a selected transgene;
iv) DNA encoding at least one persistence factor; and
v) a third negatively-charged backbone having a plurality of attached biological agents, or a negatively-charged biological agent.

In a related aspect, as described herein, in some embodiments or compositions of this invention, the positively charged backbone or carrier may be used alone to provide transdermal delivery of certain types of substances. Combinations of biologically active agents as described herein such as, for example, combinations of insulin, botulinum toxin, proteins which do not therapeutically alter blood glucose levels, antigens suitable for immunization, or non-protein non-nucleic acid agents, can also be employed in these compositions.

The negatively-charged backbones, when used to carry the imaging moieties, targeting moieties and therapeutic agents, can be a variety of backbones (similar to those described above) having multiple groups carrying a negative charge at physiological pH. Alternately, the imaging moieties, targeting moieties and therapeutic agents with sufficient surface negatively charged moieties will not require attachment of an additional backbone for ionic complex with the positively-charged backbones as will be readily apparent to one skilled in the art. Sufficient in this context implies that a suitable density of negatively-charged groups is present on the surface of the imaging moieties, targeting moieties or therapeutic agents to afford an ionic bond with the positively-charged backbones described above. In these cases, the substance or a derivative thereof have sufficient negative charge to associate with the positively charged carriers of the present invention non-covalently. The term “sufficient” in this context can be determined for example by a change in particle sizing or functional spectrophotometry versus the components alone. Suitable negatively-charged groups are carboxylic acids, phosphinic, phosphonic or phosphoric acids, sulfinic or sulfonic acids, and the like. In some embodiments, the negatively-charged backbone will be an oligonucleotide. In other embodiments, the negatively-charged backbone is an oligosaccharide (e.g., dextran). In still other embodiments, the negatively-charged backbone is a polypeptide (e.g., poly glutamic acid, poly aspartic acid, or a polypeptide in which glutamic acid or aspartic acid residues are interrupted by uncharged amino acids). The moieties described in more detail below (imaging moieties, targeting agents, and therapeutic agents) can be attached to a backbone having these pendent groups, typically via ester linkages. Alternatively, amino acids which interrupt negatively-charged amino acids or are appended to the terminus of the negatively-charged backbone, can be used to attach imaging moieties and targeting moieties via, for example, disulfide linkages (through a cysteine residue), amide linkages, ether linkages (through serine or threonine hydroxyl groups) and the like. Alternately, the imaging moieties and targeting moieties can themselves be small anions in the absence of a negatively charged polymer. Alternately, the imaging moieties, targeting moieties and therapeutic agents can be themselves covalently modified to afford sufficient surface negatively charged moieties for ionic complex with the positively-charged backbones as will be readily apparent to one skilled in the art. In both of these cases, the substance or a derivative thereof have sufficient negative charge to associate with the positively charged carriers of the present invention non-covalently. The term “sufficient” in this context refers to an association that can be determined for example by change in particle sizing or functional spectrophotometry versus the components alone.

Imaging Moieties

A variety of diagnostic or imaging moieties are useful in the present invention and are present in an effective amount that will depend on the condition-being diagnosed or imaged, the route of administration, the sensitivity of the agent and device used for detection of the agent, and the like.

Examples of suitable imaging or diagnostic agents include radiopaque contrast agents, paramagnetic contrast agents, superparamagnetic contrast agents, optical imaging moieties, CT contrast agents and other contrast agents. For example, radiopaque contrast agents (for X-ray imaging) will include inorganic and organic iodine compounds (e.g., diatrizoate), radiopaque metals and their salts (e.g., silver, gold, platinum and the like) and other radiopaque compounds (e.g., calcium salts, barium salts such as barium sulfate, tantalum and tantalum oxide). Suitable paramagnetic contrast agents (for MR imaging) include gadolinium diethylene triaminepentaacetic acid (Gd-DTPA) and its derivatives, and other gadolinium, manganese, iron, dysprosium, copper, europium, erbium, chromium, nickel and cobalt complexes, including complexes with 1,4,7,10-tetraazacyclododecane-N,N′,N″,N′″-tetraacetic acid (DOTA), ethylenediaminetetraacetic acid (EDTA), 1,4,7,10-tetraazacyclododecane-N,N′,N″-triacetic acid (DO3A), 1,4,7-triazacyclononane-N,N′,N″-triacetic acid (NOTA), 1,4,8,11-tetraazacyclotetradecane-N,N′,N″,N′″-tetraacetic acid (TETA), hydroxybenzylethylenediamine diacetic acid (HBED) and the like. Suitable superparamagnetic contrast agents (for MR imaging) include magnetites, superparamagnetic iron oxides, monocrystalline iron oxides, particularly complexed forms of each of these agents that can be attached to a negatively charged backbone. Still other suitable imaging agents are the CT contrast agents including iodinated and noniodinated and ionic and nonionic CT contrast agents, as well as contrast agents such as spin-labels or other diagnostically effective agents. Suitable optical imaging agents include for example the group consisting of Cy3, Cy3.5, Cy5, Cy5.5, Cy7, Cy7.5, Oregon green 488, Oregon green 500, Oregon, green 514, Green fluorescent protein, 6-FAM, Texas Red, Hex, TET, and HAMRA.

Other examples of diagnostic agents include marker genes that encode proteins that are readily detectable when expressed in a cell, including, but not limited to, β-galactosidase, green fluorescent protein, blue fluorescent protein, luciferase, and the like. A wide variety of labels may be employed, such as radionuclides, fluors, enzymes, enzyme substrates, enzyme cofactors, enzyme inhibitors, ligands (particularly haptens), and the like. Still other useful substances are those labeled with radioactive species or components, such as ⁹⁹mTc glucoheptonate.

The election to attach an imaging moiety to a negatively charged backbone will depend on a variety of conditions. Certain imaging agents are neutral at physiological pH and will preferably be attached to a negatively-charged backbone or covalently modified to include sufficient negatively-charged moieties above to retain a complex with the positively-charged carrier. Other imaging agents carry sufficient negative charge to retain complex with the positively-charged carrier, even in the absence of a negatively-charged backbone. In these cases, the substance or a derivative thereof have sufficient negative charge to associate with the positively charged carriers of the present invention non-covalently. The term “sufficient” in this context refers to an association that can be determined for example by change in particle sizing or functional spectrophotometry versus the components alone. Examples of such negatively-charged imaging moieties include phosphate ion (useful for magnetic resonance imaging).

Targeting Agents

A variety of targeting agents are useful in the compositions described herein. Typically, the targeting agents are attached to a negatively-charged backbone as described for the imaging moieties above. In certain embodiments, the targeting agents and the imaging moieties are structurally and/or chemically distinct. For example, the imaging moieties and targeting agents are both not phosphate. Generally, the targeting agents can be any element that makes it possible to direct the transfer of a nucleic acid, therapeutic agent or another component of the composition to a particular site or to alter the tropism of the complex relative to that of the complex without the targeting agent. The targeting agent can be an extracellular targeting agent, which allows, for example, a nucleic acid transfer to be directed towards certain types of cells or certain desired tissues (tumor cells, liver cells, hematopoietic cells, and the like). Such an agent can also be an intracellular targeting agent, allowing a therapeutic agent to be directed towards particular cell compartments (e.g, mitochondria, nucleus, and the like). The agent most simply can also be a small anion which, by virtue or changing net charge distribution alters the tropism of the complex from more highly negative cell surfaces and extracellular matrix components to a wider variety of cells or even specifically away from the most highly negative surfaces.

The targeting agent or agents are preferably linked, covalently or non-covalently, to a negatively-charged backbone according to the invention. According to a preferred mode of the invention, the targeting agent is covalently attached to an oligonucleic acid, polyaspartate, sulfated or phosphorylated dextran and the like that serves as a negatively-charged backbone component, preferably via a linking group. Methods of attaching targeting agents (as well as other biological agents) to nucleic acids are well known to those of skill in the art using, for example, heterobifunctional linking groups (see Pierce Chemical Catalog). In one group of embodiments, the targeting agent is a fusogenic peptide for promoting cellular transfection, that is to say for favoring the passage of the composition or its various elements across membranes, or for helping in the egress from endosomes or for crossing the nuclear membrane. The targeting agent can also be a cell receptor ligand for a receptor that is present at the surface of the cell type, such as, for example, a sugar, transferrin, insulin or asialo-orosomucoid protein. Such a ligand may also be one of intracellular type, such as a nuclear location signal (nls) sequence which promotes the accumulation of transfected DNA within the nucleus.

Other targeting agents useful in the context of the invention, include sugars, peptides, hormones, vitamins, cytokines, oligonucleic acids, small anions, lipids or sequences or fractions derived from these elements and which allow specific binding with their corresponding receptors. Preferably, the targeting agents are sugars and/or peptides such as antibodies or antibody fragments, cell receptor ligands or fragments thereof, receptors or receptor fragments, and the like. More preferably, the targeting agents are ligands of growth factor receptors, of cytokine receptors, or of cell lectin receptors or of adhesion protein receptors. The targeting agent can also be a sugar which makes it possible to target lectins such as the asialoglycoprotein receptors, or alternatively an antibody Fab fragment which makes it possible to target the Fc fragment receptor of immunoglobulins.

In still other embodiments, a targeting agent is used in the absence of a negatively-charged backbone. In this group of embodiments, the targeting agent carries sufficient negatively charged moieties to retain an ionic complex with the positively-charged carrier described above. In these cases, the substance or a derivative thereof have sufficient negative charge to associate with the positively charged carriers of the present invention non-covalently. The term “sufficient” in this context refers to an association that can be determined for example by change in particle sizing or functional spectrophotometry versus the components alone. Suitable negatively-charged targeting agents for this group of embodiments are protein-based targeting agents having a net negative charge at physiological pH, as well as targeting agents that can facilitate adhesion to a particular cell surface, such as small polyanions including for example phosphate, aspartate and citrate which can for example change targeting based upon net surface charge of the cell to be targeted.

In the compositions of the present invention, the nucleic acid can be either a deoxyribonucleic acid or a ribonucleic acid, and can comprise sequences of natural or artificial origin. More particularly, the nucleic acids used herein can include genomic DNA, cDNA, mRNA, tRNA, rRNA, hybrid sequences or synthetic or semi-synthetic sequences. These nucleic acids can be of human, animal, plant, bacterial, viral, etc. origin. Additionally, the nucleic acids can be obtained by any technique known to those skilled in the art, and in particular by the screening of banks, by chemical synthesis or by mixed methods including the chemical or enzymatic modification of sequences obtained by the screening of banks. Still further, the nucleic acids can be incorporated into vectors, such as plasmid vectors.

The deoxyribonucleic acids used in the present invention can be single- or double-stranded. These deoxyribonucleic acids can also code for therapeutic genes, sequences for regulating transcription or replication, antisense sequences, regions for binding to other cell components, etc. Suitable therapeutic genes are essentially any gene which codes for a protein product having a therapeutic effect. The protein product thus encoded may be a protein, polypeptide, a peptide, or the like. The protein product can, in some instances, be homologous with respect to the target cell (that is to say a product which is normally expressed in the target cell when the latter exhibits no pathology). In this manner, the use of suitable nucleic acids can increase the expression of a protein, making it possible, for example, to overcome an insufficient expression in the cell. Alternatively, the present invention provides compositions and methods for the expression of a protein which is inactive or weakly active due to a modification, or alternatively of overexpressing the protein. The therapeutic gene may thus code for a mutant of a cell protein, having increased stability, modified activity, etc. The protein product may also be heterologous with respect to the target cell. In this case, an expressed protein may, for example, make up or provide an activity which is deficient in the cell, enabling it to combat a pathology or to stimulate an immune response.

More particularly, nucleic acids useful in the present invention are those that code for enzymes, blood derivatives, hormones, lymphokines, interleukins, interferons, TNF, growth factors, neurotransmitters or their precursors or synthetic enzymes, or trophic factors: BDNF, CNTF, NGF, IGF, GMF, aFGF, bFGF, VEGF, NT3, NT5, HARP/pleiotrophin; the proteins involved in the metabolism of lipids, of apolipoprotein-types selected from apolipoproteins A-I, A-II, A-IV, B, C-I, C-II, C-III, D, E, F, G, H, J and apo(a), metabolic enzymes such as, for example, lipoprotein lipase, hepatic lipase, lecithin cholesterol acyltransferase, 7-α-cholesterol hydroxylase, phosphatidic acid phosphatase, or lipid transfer proteins such as cholesterol ester transfer protein and phospholipid transfer protein, a protein for binding HDLs or a receptor selected from, for example, LDL receptors, chylomicron-remnant receptors and scavenger receptors, dystrophin or minidystrophin, GAX protein, CFTR protein associated with mucoviscidosis, tumor-suppressant genes: p53, Rb, Rap1A, DCC, k-rev; protein factors involved in coagulation: factors VII, VIII, IX; or the nucleic acids can be those genes involved in DNA repair, suicide genes (thymidine kinase, cytosine deaminase), genes encoding thrombomodulin, α1-antitrypsin, tissue plasminogen activator, superoxide dismutase, elastase, matrix metalloproteinase, and the like.

The therapeutic genes useful in the present invention can also be an antisense sequence or a gene whose expression in the target cell makes it possible to control the expression of genes or the transcription of cellular mRNA. Such sequences can, for example, be transcribed in the target cell into complementary RNA of cellular mRNA and thus block their translation into protein, according to the technique described in patent EP 140,308. The antisense sequences also comprise the sequences coding for ribozymes which are capable of selectively destroying target RNA (see EP 321,201).

As indicated above, the biologically active agent may also comprise one or more antigenic peptides that are capable of generating an immune response in humans or animals. In this particular embodiment, the invention thus makes it possible to produce either vaccines or immunotherapeutic treatments applied to humans or to animals, in particular against microorganisms, viruses or cancers. They may in particular be antigenic peptides specific for Epstein-Barr virus, for HIV virus, for hepatitis B virus (see EP 185,573), for pseudo-rabies virus or alternatively specific for tumors (see EP 259,212).

Preferably, the nucleic acid also comprises sequences that allow the expression of the therapeutic gene and/or of the gene coding for the antigenic peptide in the desired cell or organ. These can be sequences that are naturally responsible for expression of the gene considered when these sequences are capable of functioning in the infected cell. The nucleic acids can also be sequences of different origin (responsible for the expression of other proteins, or even synthetic proteins). In particular, the nucleic acids can contain promoter sequences for eukaryotic or viral genes. For example, the promoter sequences can be those derived from the genome of the cell which it is desired to infect. Similarly, the promoter sequences can be derived from the genome of a virus, e.g., the promoters of genes ElA, MLP, CMV, RSV, etc. In addition, these expression sequences may be modified by addition of activation sequences, regulation sequences, etc.

Moreover, the nucleic acid may also contain, in particular upstream of the therapeutic gene, a signal sequence which directs the therapeutic product synthesized into the secretion pathways of the target cell. This signal sequence may be the natural signal sequence of the therapeutic product, but it may also be any other functional signal sequence, or an artificial signal sequence.

DNA Encoding at Least One Persistence Factor

In some embodiments, the composition will also comprise DNA encoding at least one persistence factor. Exemplary of such DNA is the DNA encoding adenoviral preterminal protein 1 (see, Lieber, et al. Nature Biotechnology 15(13):1383-1387 (1997). Adenoviral preterminal protein 1 or the nucleic acid encoding it can be provided in cis- or trans- to the nucleic acid sequence encoding the desired therapeutic transgene. When provided in this manner, the preterminal protein 1 or sequence preserves the therapeutic nucleic acid as a stable nuclear episome and thus prevents loss of the therapeutic nucleic acid and prevents late decreases in therapeutic protein expression.

Biological Agents

A variety of biological agents, including both therapeutic and cosmeceutical agents, are useful in the present invention and are present in an effective amount that will depend on the condition being treated, prophylactically or otherwise, the route of administration, the efficacy of the agent and patient's size and susceptibility to the treatment regimen.

Suitable therapeutic agents that can be attached to a negatively charged backbone can be found in essentially any class of agents, including, for example, analgesic agents, anti-asthmatic agents, antibiotics, antidepressant agents, anti-diabetic agents, antifungal agents, antiemetics, antihypertensives, anti-impotence agents, anti-inflammatory agents, antineoplastic agents, anti-HIV agents, antiviral agents, anxiolytic agents, contraception agents, fertility agents, antithrombotic agents, prothrombotic agents, hormones, vaccines, immunosuppressive agents, vitamins and the like. Alternately, sufficient negatively charged groups can be introduced into the therapeutic agent to afford ionic complex with the positively charged backbones described above. Many suitable methods such as phosphorylation or sulfation exist as will be readily apparent to one skilled in the art.

Further, certain agents themselves possess adequate negatively-charged moieties to associate with the positively charged carrier described above and do not require attachment to a negatively charged backbone. In these cases, the substance or a derivative thereof have sufficient negative charge to associate with the positively charged carriers of the present invention non-covalently. The term “sufficient” in this context refers to an association that can be determined for example by change in particle sizing or functional spectrophotometry versus the components alone.

Suitable cosmeceutic agents include, for example, epidermal growth factor (EGF), as well as human growth hormone, antioxidants, and botulinum toxin. In the context of this invention, the term “botulinum toxin” includes not only botulinum serotypes A, B, C, D, E, F, and G, but also fragments thereof having botulinum light-chain activity.

More particularly, therapeutic agents useful in the present invention include such analgesics as lidocaine, novocaine, bupivacaine, procaine, tetracaine, benzocaine, cocaine, mepivacaine, etidocaine, proparacaine ropivacaine, prilocaine and the like; anti-asthmatic agents such as azelastine, ketotifen, traxanox, corticosteroids, cromolyn, nedocromil, albuterol, bitolterol mesylate, pirbuterol, salmeterol, terbutyline, theophylline and the like; antibiotic agents such as neomycin, streptomycin, chloramphenicol, norfloxacin, ciprofloxacin, trimethoprim, sulfamethyloxazole, the β-lactam antibiotics, tetracycline, and the like; antidepressant agents such as nefopam, oxypertine, imipramine, trazadone and the like; anti-diabetic agents such as biguanidines, sulfonylureas, and the like; antiemetics and antipsychotics such as chloropromazine, fluphenazine, perphenazine, prochlorperazine, promethazine, thiethylperazine, triflupromazine, haloperidol, scopolamine, diphenidol, trimethobenzamide, and the like; neuromuscular agents such as atracurium mivacurium, rocuronium, succinylcholine, doxacurium, tubocurarine, and botulinum toxin (BTX); antifungal agents such as amphotericin B, nystatin, candicidin, itraconazole, ketoconazole, miconazole, clotrimazole, fluconazole, ciclopirox, econazole, naftifine, terbinafine, griseofulvin, ciclopirox and the like; antihypertensive agents such as propanolol, propafenone, oxyprenolol, nifedipine, reserpine and the like; anti-impotence agents such as nitric oxide donors and the like; anti-inflammatory agents including steroidal anti-inflammatory agents such as cortisone, hydrocortisone, dexamethasone, prednisolone, prednisone, fluazacort, and the like, as well as non-steroidal anti-inflammatory agents such as indomethacin, ibuprofen, ramifenizone, prioxicam and the like; antineoplastic agents such as adriamycin, cyclophosphamide, actinomycin, bleomycin, duanorubicin, doxorubicin, epirubicin, mitomycin, rapamycin, methotrexate, fluorouracil, carboplatin, carmustine (BCNU), cisplatin, etoposide, interferons, phenesterine, taxol (including analogs and derivatives), caniptothecin and derivatives thereof, vinblastine, vincristine and the like; anti-HIV agents (e.g., antiproteolytics); antiviral agents such as amantadine, methisazone, idoxuridine, cytarabine, acyclovir, famciclovir, ganciclovir, foscamet, sorivudine, trifluridine, valacyclovir, cidofovir, didanosine, stavudine, zalcitabine, zidovudine, ribavirin, rimantatine and the like; anxiolytic agents such as dantrolene, diazepam and the like; COX-2 inhibitors; contraception agents such as progestogen and the like; anti-thrombotic agents such as GPIIb/IIIa inhibitors, tissue plasminogen activators, streptokinase, urokinase, heparin and the like; prothrombotic agents such as thrombin, factors V, VII, VIII and the like; hormones such as insulin, growth hormone, prolactin, EGF (epidermal growth factor) and the like; immunosuppressive agents such as cyclosporine, azathioprine, mizorobine, FK506, prednisone and the like; angiogenic agents such as VEGF (vascular endothelial growth factor); vitamins such as A, D, E, K and the like; and other therapeutically or medicinally active agents. See, for example, GOODMAN & GILMAN'S THE PHARMACOLOGICAL BASIS OF THERAPEUTICS, Ninth Ed. Hardman, et al., eds. McGraw-Hill, (1996).

In the most preferred embodiments, the biological agent is selected from insulin, botulinum toxin, VEGF, antigens for immunization, and antifungal agents.

As noted above for the targeting agents and imaging agents, certain biological or cosmeceutical agents can be used in the absence of a negatively-charged backbone. Such biological or cosmeceutical agents are those that generally carry a net negative charge at physiological pH to retain complex with the positively-charged carrier. Examples include botulinum toxin (a large MW protein), insulin (a small MW protein), antigens for immunization, which can range from very small to very large and typically include proteins or glycoproteins, and many antifungal agents. In these cases, the substance or a derivative thereof has a sufficient negative charge to associate with the positively charged carriers of the present invention non-covalently. The term “sufficient” in this context refers to an association that can be determined, for example, by change in particle sizing or functional spectrophotometry versus the components alone.

Negatively-Charged Backbones Having Attached Imaging Moieties, Targeting Agents or Therapeutic Agents

For three of the above groups of components, including imaging moieties, targeting agents and therapeutic agents, the individual compounds can be attached to a negatively charged backbone, covalently modified to introduce negatively-charged moieties, or employed directly if the compound contains sufficient negatively-charged moieties to confer ionic binding to the positively charged backbone described above. When necessary, typically, the attachment is via a linking group used to covalently attach the particular agent to the backbone through functional groups present on the agent as well as the backbone. A variety of linking groups are useful in this aspect of the invention. See, for example, Hermanson, Bioconjugate Techniques, Academic Press, San Diego, Calif. (1996); Wong, S. S., Ed., Chemistry of Protein Conjugation and Cross-Linking, CRC Press, Inc., Boca Raton, Fla. (1991); Senter, et al., J. Org. Chem. 55:2975-78 (1990); and Koneko, et al., Bioconjugate Chem. 2:133-141 (1991).

In some embodiments, the therapeutic, diagnostic or targeting agents will not have an available functional group for attaching to a linking group, and can be first modified to incorporate, for example, a hydroxy, amino, or thiol substituent. Preferably, the substituent is provided in a non-interfering portion of the agent, and can be used to attach a linking group, and will not adversely affect the function of the agent.

In yet another -aspect, the present invention provides compositions comprising a non-covalent complex of a positively-charged backbone having at least one attached efficiency group and at least one nucleic acid member selected from the group consisting of RNA, DNA, ribozymes, modified oligonucleic acids and cDNA encoding a selected transgene. In this aspect of the invention, the positively-charged backbone can be essentially any of the positively-charged backbones described above, and will also comprise (as with selected backbones above) at least one attached efficiency group. Suitable efficiency groups include, for example, (Gly)ₙ₁-(Arg)ₙ₂ wherein the subscript n1 is an integer of from 3 to about 5, and the subscript n2 is independently an odd integer of from about 7 to about 17 or TAT domains. Additionally, the nucleic acids useful in this aspect of the invention are the same as have been described above.

Transdermal Delivery of Insulin and Certain Larger Molecules

It has been found that the positively charged carriers above can be used for transdermal delivery of insulin and certain other biologically active agents which do not therapeutically alter blood glucose levels, such as proteins having a molecular weight of about 50,000 and above, for instance, botulinum toxin (BTX), or for other biologically active agents such as a therapeutic nucleic acid-based agent, a non-protein non-nucleic acid therapeutic agent such as certain antifungal agents or alternately an agent for immunization. The use of the positively charged carrier enables transmittal of the protein or marker gene both into and out of skin cells, and delivery of it in an effective amount and active form to an underlying tissue. For example, insulin may be delivered through the skin into underlying capillaries for transport through the body without the need for injection. Botulinum toxin can be delivered to muscles underlying or glandular structures within the skin in an effective amount to produce paralysis, produce relaxation, alleviate contractions, prevent or alleviate spasms, reduce glandular output or provide other desired effects. Local delivery in this manner could afford dosage reductions, reduce toxicity and allow more precise dosage optimization for desired effects relative to injectable or implantable materials, particularly in the case of botulinum toxin. This embodiment may include a quantity of a small preferably polyvalent anion, for example, phosphate, aspartate, or citrate, or may be carried out in the substantial absence of such a polyanion. In all aspects of the present invention, the association between the carrier and the biologically active agent is by non-covalent interaction, which can include, for example, ionic interactions, hydrogen bonding, van der Waals forces, or combinations thereof.

The term “botulinum toxin” as used herein is meant to refer to any of the known serotypes of botulinum toxin, whether produced by the bacterium or by recombinant techniques, as well as any such types that may be subsequently discovered including engineered variants or fusion proteins. As mentioned above, at the present time, seven immunologically distinct botulinum neurotoxins have been characterized, namely botulinum neurotoxin serotypes A, B, C, D, E, F and G, each of which is distinguished by neutralization with type-specific antibodies. The botulinum toxin serotypes are available from Sigma-Aldrich and from Metabiologics, Inc. (Madison, Wis.), as well as from other sources. The different serotypes of botulinum toxin vary in the animal species that they affect and in the severity and duration of the paralysis they evoke. At least two types of botulinum toxin, types A and B, are currently available commercially in formulations for treatment of certain conditions. Type A, for example, is contained in preparations of Allergan having the trademark BOTOX® and of Ipsen having the trademark DYSPORT®, and type B is contained in preparations of Elan having the trademark MYOBLOC®.

The botulinum toxin used in the compositions of this invention can be a botulinum toxin derivative, that is, a compound that has botulinum toxin activity but contains one or more chemical or functional alterations on any part or on any chain relative to naturally occurring or recombinant native botulinum toxins. For instance, the botulinum toxin may be a modified neurotoxin, that is a neurotoxin which has at least one of its amino acids deleted, modified or replaced, as compared to a native, or the modified neurotoxin can be a recombinant produced neurotoxin or a derivative or fragment thereof. For instance, the botulinum toxin may be one that has been modified in a way that, for instance, enhances its properties or decreases undesirable side effects, but that still retains the desired botulinum toxin activity. The botulinum toxin may be any of the botulinum toxin complexes produced by the bacterium, as described above. Alternatively the botulinum toxin may be a toxin prepared using recombinant or synthetic chemical techniques, e.g. a recombinant peptide, a fusion protein, or a hybrid neurotoxin, for example prepared from subunits or domains of different botulinum toxin serotypes (see U.S. Pat. No. 6,444,209, for instance). The botulinum toxin may also be a portion of the overall molecule that has been shown to possess the necessary botulinum toxin activity, and in such case may be used per se or as part of a combination or complex molecule, for instance a fusion protein. Alternately, a portion of the toxin may be used directly with the positively charged backbones described herein with or without targeting moieties since the positively charged backbone allows cellular internalization even in the absence of the native BTX binding, targeting, or internalization domains. Alternatively, the botulinum toxin may be in the form of a botulinum toxin precursor, which may itself be non-toxic, for instance a nontoxic zinc protease that becomes toxic on proteolytic cleavage.

This invention also contemplates the general use of combinations and mixtures of botulinum toxins, though due to their differing nature and properties, mixtures of botulinum toxin serotypes are not generally administered at this time.

Similarly, the term “insulin” includes insulin extracted from natural sources, as well as insulin that may be obtained synthetically, via chemical or recombinant means. The insulin also may be in a modified form, or in the form of, e.g. a recombinant peptide, a fusion protein, or a hybrid molecule, or the insulin in a particular case may be a portion of the insulin molecule that possesses the necessary activity. The same is true of other proteins that may be used in these particular transdermal compositions and methods, particularly antigens for immunization, which can vary widely in physiochemical properties. Likewise non-protein non-nucleic acid therapeutic agents, including antifungal agents, may be obtained from natural sources or may be synthesized.

Compositions of this invention are preferably in the form of products to be applied to the skin or epithelium of subjects or patients, i.e. humans or other mammals in need of the particular treatment. The term “in need” is meant to include both pharmaceutical and health-related needs as well as needs that tend to be more cosmetic, aesthetic, or subjective. The botulinum toxin compositions may also be used, for example, for altering or improving the appearance of facial tissue.

Through the use of the positively charged carriers of this invention, a botulinum toxin can be administered transdermally to a subject for treating conditions such as undesirable facial muscle or other muscular spasms, hyperhidrosis, acne, or conditions elsewhere in the body in which relief of muscular ache or spasms is desired. The botulinum toxin is administered topically for transdermal delivery to muscles or to other skin-associated structures. The administration may be made, for example, to the legs, shoulders, back including lower back, axilla, palms, feet, neck, groin, dorsa of the hands or feet, elbows, upper arms, knees, upper legs, buttocks, torso, pelvis, or any other part of the body where administration of the botulinum toxin is desired.

Administration of botulinum toxin may also be carried out to treat other conditions, including treating of neurologic pain, prevention or reduction of migraine headache or other headache pain, prevention or reduction of acne, prevention or reduction of dystonia or dystonic contractions whether subjective or clinical, prevention or reduction of symptoms associated with subjective or clinical hyperhidrosis, reducing hypersecretion or sweating, reducing or enhancing immune response, or treatment of other conditions for which administration of botulinum toxin by injection has been suggested or performed. Administration of botulinum toxin, other therapeutic proteins which do not have a therapeutic effect on blood glucose levels, other antigens useful for immunization described herein, or other non-nucleic acid non-protein therapeutic agents for instance, the complexed botulinum toxin, may also be carried out for immunization-related purposes. Alternately, the complex can be prepared and applied topically to enhance an immune response, for example to provide immunizations respecting various proteins, for example, for childhood immunizations without injections or immunization against various environmental hazards. Surprisingly, administration of botulinum toxin or other therapeutic proteins, described herein may also be carried out to reduce immune responses. The present invention allows BIX and other protein to be delivered by an altered route of administration and changes the complex antigen presentation of the agent and may thus be useful to reduce immune response to antigens to that protein, and thus facilitate repeat administration without immune-related reduction in activity.

In general, the compositions are prepared by mixing the insulin, botulinum toxin, or other biologically active agent such as for example, a therapeutic protein which does not therapeutically alter blood glucose levels, a therapeutic nucleic acid-based agent, a non-protein non-nucleic acid therapeutic agent or alternately an agent for immunization to be administered with the positively charged carrier, and usually with one or more additional pharmaceutically acceptable carriers or excipients. In their simplest form they may contain a simple aqueous pharmaceutically acceptable carrier or diluent, such as saline, which may be buffered. However, the compositions may contain other ingredients typical in topical pharmaceutical or cosmeceutical compositions, that is, a dermatologically or pharmaceutically acceptable carrier, vehicle or medium, i.e. a carrier, vehicle or medium that is compatible with the tissues to which they will be applied. The term “dermatologically or pharmaceutically acceptable,” as used herein, means that the compositions or components thereof so described are suitable for use in contact with these tissues or for use in patients in general without undue toxicity, incompatibility, instability, allergic response, and the like. As appropriate, compositions of the invention may comprise any ingredient conventionally used in the fields under consideration, and particularly in cosmetics and dermatology. In all aspects of the present invention, the association between the carrier and the biologically active agent is by non-covalent interaction, which can include, for example, ionic interactions, hydrogen bonding, van der Waals forces, or combinations thereof.

The compositions may be pre-formulated or may be prepared at the time of administration, for example, by providing a kit for assembly at or prior to the time of administration. Alternatively, as mentioned above, the botulinum toxin or other therapeutic protein and the positively charged backbone or carrier may be administered in separate form to the patient, for example by providing a kit that contains a skin patch or other dispensing device containing the therapeutic protein and a liquid, gel, cream or the like that contains the positively charged carrier (and optionally other ingredients). In that particular embodiment the combination is administered by applying the liquid or other composition containing the carrier to the skin, followed by application of the skin patch or other device.

The compositions of the invention are applied so as to administer an effective amount of the insulin, botulinum toxin, or other beneficial substance. For transdermal delivery the term “effective amount” refers to any composition or method that provides greater transdermal delivery of the biologically active agent relative to the agent in the absence of the carrier. For botulinum toxin, the term “effective amount” as used herein means an amount of a botulinum toxin as defined above that is sufficient to produce the desired muscular paralysis or other effect, but that implicitly is a safe amount, i.e. one that is low enough to avoid serious side effects. Desired effects include the relaxation of certain muscles with the aim of, for instance, decreasing the appearance of fine lines and/or wrinkles, especially in the face, or adjusting facial appearance in other ways such as widening the eyes, lifting the corners of the mouth, or smoothing lines that fan out from the upper lip, or the general relief of muscular tension. The last-mentioned effect, general relief of muscular tension, can be accomplished in the face or elsewhere, for example in the back or legs. For insulin, the term “effective amount” similarly means an amount of insulin that is sufficient to produce the desired effect, namely decrease of glucose in the patient or subject's blood. For antigens,“effective amount” refers to an amount sufficient to allow a subject to mount an immune response to the antigen after application or a series of applications of the antigen. For antifungal agents, “effective amount” refers to an amount sufficient to reduce symptoms or signs of fungal infection. For other biologically active agents which do not therapeutically alter blood glucose levels, “effective amount” refers to an amount sufficient to exert the defined biologic or therapeutic effect characterized for that agent in for example the Physicians' Desk Reference or the like without inducing significant toxicity. The invention specifically excludes antibody fragments which do not have biological activity other than only binding a specific antigen when the term “therapeutic” or “biologically active protein” is employed. Since antigens suitable for immunization have other biological activities such as mounting an immune response, these remain included in the appropriate aspects of this invention, however. Moreover, agents that have a biological activity or a therapeutic effect by binding a specific antigen, thereby blocking ligand binding or altering the conformation of the antigen are included in this invention.

The compositions may contain an appropriate effective amount of the insulin, botulinum toxin, or other biologically active agent such as for example, a therapeutic protein which does not therapeutically alter blood glucose levels, a therapeutic nucleic acid-based agent, a non-protein non-nucleic acid therapeutic agent or alternately an agent for immunization, for application as a single-dose treatment, or may be more concentrated, either for dilution at the place of administration or for use in multiple applications. In general, compositions containing botulinum toxin or other biologically active agent such as for example, a therapeutic protein which does not therapeutically alter blood glucose levels or a therapeutic nucleic acid-based agent will contain from about 1×10⁻²⁰ to about 25 weight % of the biologically active agent and from about 1×10⁻¹⁹ to about 30 weight % of the positively charged carrier. In general, compositions containing a non-protein non-nucleic acid therapeutic agent or alternately an agent for immunization will contain from about 1×10⁻¹⁰ to about 49.9 weight % of the antigen and from about 1×10⁻⁹ to about 50 weight % of the positively charged carrier. In general, in a form suitable for application to the subject, the compositions of the invention will contain from about 0.001 to about 10,000 preferably from about 0.01 to about 1,000 IU/g of a composition comprising botulinum toxin and a positively charged carrier molecule as described herein. The ratio of carrier: botulinum toxin preferably ranges from about 10:1 to about 1.01:1 and more preferably from about 6:1 to about 1.5:1 respectively. The amount of carrier molecule or the ratio of it to the botulinum toxin will depend on which carrier is chosen for use in the composition in question. The appropriate amount or ratio of carrier molecule in a given case can readily be determined, for example, by conducting one or more experiments such as those described below.

The compositions of this invention allow for the delivery of a more pure botulinum toxin with higher specific activity potentially improved pharmacokinetics. In addition, the positively charged carrier reduces the need for foreign accessory proteins (e.g., human serum albumin ranging from 400-600 mg or recombinant serum albumin ranging from 250-500 mg) and polysaccharide stabilizers and can afford beneficial reductions in immune responses to the BTX. In addition, the compositions are suitable for use in physiologic environments with pH ranging from 4.5 to 6.3, and may thus have such a pH. The compositions may be stored preferably either at room temperature or under refrigerated conditions.

The botulinum toxin-containing compositions or devices will generally be applied so as to provide the botulinum toxin at a dose of from about 1 U to abut 20,000 U, preferably from about 1 U to about 10,000 U, of botulinum toxin per cm² of skin, per application. Higher dosages within these ranges could preferably be employed in conjunction with controlled release materials, for instance, or allowed a shorter dwell time on the skin prior to removal.

In the case of insulin, the compositions of the invention will contain from about 0.011 U to about 5000 U, preferably from about 0.1 U to about 500 U/gram. A composition comprising a form of insulin and a positively charged carrier molecule as described herein preferably ranges from about 30:1 to about 1.01:1 and more preferably from about 6:1 to about 1.25:1 of insulin:carrier, respectively. Likewise, the amount of carrier molecule or the ratio of it to the insulin will depend on which carrier is chosen for use in the composition in question.

In terms of their form, compositions of this invention may include solutions, emulsions (including microemulsions), suspensions, creams, lotions, gels, powders, or other typical solid or liquid compositions used for application to skin and other tissues where the compositions may be used. Such compositions may contain, in addition to the botulinum toxin, insulin or other biologically active agent, and the carrier molecule, other ingredients typically used in such products, such as antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals. In all aspects of the present invention, the association between the carrier and the biologically active agent is by non-covalent interaction, which can include, for example, ionic interactions, hydrogen bonding, van der Waals forces, or combinations thereof

Compositions according to this invention may be in the form of controlled-release or sustained-release compositions, wherein the insulin, botulinum toxin, or other substance to be delivered and the carrier are encapsulated or otherwise contained within a material such that they are released onto the skin in a controlled manner over time. The substance to be delivered and the carrier may be contained within matrixes, liposomes, vesicles, microcapsules, microspheres and the like, or within a solid particulate material, all of which is selected and/or constructed to provide release of the substance or substances over time. The therapeutic substance and the carrier may be encapsulated together (e.g., in the same capsule) or separately (in separate capsules).

Administration of the compositions of this invention to a subject is, of course, another aspect of the invention. In the case of botulinum toxin, most preferably the compositions are administered by or under the direction of a physician or other health professional. They may be administered in a single treatment or in a series of periodic treatments over time. For transdermal delivery of botulinum toxin for the purposes mentioned above, a composition as described above is applied topically to the skin at a location or locations where the effect is desired. Because of its nature, most preferably the amount of botulinum toxin applied should be applied with care, at an application rate and frequency of application that will produce the desired result without producing any adverse or undesired results.

In the case of insulin, for hospitalized patients or in-office treatments, the administration will be carried out by or under the direction of a health care professional, but otherwise is likely to be performed by the patient. Administration by skin patches and the like, with controlled release and/or monitoring is likely to be a common method, so the insulin-containing compositions of this invention often will be provided as contained in a skin patch or other device. In the case of antigens suitable for immunizations, most preferably the compositions are administered by or under the direction of a physician or other health professional. They may be administered in a single treatment or in a series of periodic treatments over time. Accordingly, sustained release compositions are also contemplated by this invention. For transdermal delivery of antigens suitable for immunizations for the purposes mentioned above, a composition as described above is applied topically to the skin or to a nail plate and surrounding skin. In the case of non-protein, non-nucleic acid therapeutics such as antifungal agents, preferably the compositions are administered under the direction of a physician or other health professional. They may be administered in a single treatment or in a series of periodic treatments over time. Sustained release compositions are also contemplated for non-protein, non-nucleic acid therapeutics. Antifungal agents may be administered to the finger nail or toe nail plate or surrounding anatomic structures using, for instance, a prosthetic nail plate, a lacquer, a nail polish with a color agent, a gel, or a combination of any or all of these. For transdermal delivery of botulinum toxin for the purposes mentioned above, a composition as described above is applied topically to the skin

Kits for administering the compositions of the inventions, either under direction of a health care professional or by the patient or subject, may also include a custom applicator suitable for that purpose. The term “custom applicator” is meant to include the means just mentioned for administering antifungal agents.

In another aspect, the invention relates to methods for the topical administration of the combination of the positively charged carrier described above with an effective amount of insulin, botulinum toxin, antigens suitable for immunization, antifungal agents or other biologically active agent such as for example, a therapeutic protein which does not therapeutically alter blood glucose levels, a therapeutic nucleic acid-based agent, or a non-protein non-nucleic acid therapeutic agent, in general. As described above, the administration can be effected by the use of a composition according to the invention that contains appropriate types and amounts of these two substances specifically carrier and biologically active agent. However, the invention also includes the administration of these two substances in combination, though not necessarily in the same composition. For example, the therapeutic or biologically active substance may be incorporated in dry form in a skin patch or other dispensing device, and the positively charged carrier may be applied to the skin surface before application of the patch so that the two act together, resulting in the desired transdermal delivery. In that sense, thus, the two substances, specifically carrier and biologically active agent, act in combination or in conjunction, or perhaps interact to form a composition or combination in situ.

Methods of Preparing the Compositions

In another aspect, the present invention provides a method for preparing a pharmaceutical composition, the method comprising combining a positively charged backbone component and at least two members selected from the group consisting of:
i) a first negatively-charged backbone having a plurality of attached imaging moieties, or alternatively a plurality of negatively-charged imaging moieties;
ii) a second negatively-charged backbone having a plurality of attached targeting agents, or alternatively a plurality of negatively-charged targeting moieties;
iii) at least one member selected from RNA, DNA, ribozymes, modified oligonucleic acids and cDNA encoding a selected transgene;
iv) DNA encoding at least one persistence factor; and
v) a third negatively-charged backbone having a plurality of attached biological agents, or a negatively-charged biological agent;
with a pharmaceutically acceptable carrier to form a non-covalent complex having a net positive charge, with the proviso that at least one of the members is selected from i), ii), iii) or v).

In a related aspect, as described herein, in some embodiments or compositions of this invention, the positively charged backbone or carrier may be used alone to provide transdermal delivery of certain types of substances. Here preferred are compositions and methods comprising a biologically active agent such as a botulinum toxin or other therapeutic protein which does not lower blood glucose containing from about 1×10⁻²⁰ to about 25 weight % of the biologically active agent and from about 1×10⁻¹⁹ to about 30 weight % of the positively charged carrier. Also preferred are compositions and methods comprising a non-nucleic acid non-protein therapeutic such as an antifungal agent or an antigen suitable for immunization containing from 1×10⁻¹⁰ to about 49.9 weight % of the antigen and from about 1×10⁻⁹ to about 50 weight % of the positively charged carrier. In all aspects of the present invention, the association between the carrier and the biologically active agent is by non-covalent interaction, which can include, for example, ionic interactions, hydrogen bonding, van der Waals forces, or combinations thereof.

The broad applicability of the present invention is illustrated by the ease with which a variety of pharmaceutical compositions can be formulated. Typically, the compositions are prepared by mixing the positively charged backbone component with the desired components of interest (e.g., DNA, targeting, imaging or therapeutic components) in ratios and a sequence to obtain compositions having a variable net positive charge. In many embodiments, the compositions can be prepared, for example, at bedside using pharmaceutically acceptable carriers and diluents for administration of the composition. Alternatively, the compositions can be prepared by suitable mixing of the components and then lyophilized and stored (typically at room temperature or below) until used or formulated into a suitable delivery vehicle.

The compositions can be formulated to provide mixtures suitable for topical, cutaneous, oral, rectal, vaginal, parenteral, intranasal, intravenous, intramuscular, subcutaneous, intraocular, transdermal, etc. administration. The pharmaceutical compositions of the invention preferably contain a vehicle which is pharmaceutically acceptable for an injectable formulation, in particular for direct injection into the desired organ, or for topical administration (to skin and/or mucous membrane). They may in particular be sterile, isotonic solutions or dry compositions, in particular freeze-dried compositions, which, by addition, depending on the case, of sterilized water or of physiological saline, allow injectable solutions to be made up. For example, the doses of nucleic acid used for the injection and the number of administrations may be adapted according to various parameters, and in particular according to the mode of administration used, the pathology concerned, the gene to be expressed, or alternatively the desired duration of the treatment.

Alternatively, when the compositions are to be applied topically, e.g. when transdermal delivery is desired, the component or components of interest can be applied in dry form to the skin, e.g. via by using a skin patch, where the skin is separately treated with the positively charged backbone or carrier. In this manner the overall composition is essentially formed in situ and administered to the patient or subject.

Methods of Using the Compositions Delivery Methods

The compositions of the present invention can be delivered to a subject, cell or target site, either in vivo or ex vivo using a variety of methods. In fact, any of the routes normally used for introducing a composition into ultimate contact with the tissue to be treated can be used. Preferably, the compositions will be administered with pharmaceutically acceptable carriers. Suitable methods of administering such compounds are available and well known to those of skill in the art, and, although more than one route can be used to administer a particular composition, a particular route can often provide a more immediate and more effective reaction than another route. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985).

Administration can be, for example, intravenous, topical, intraperitoneal, subdermal, subcutaneous, transcutaneous, intramuscular, oral, intra-joint, parenteral, intranasal, or by inhalation. Suitable sites of administration thus include, but are not limited to, the skin, bronchium, gastrointestinal tract, eye and ear. The compositions typically include a conventional pharmaceutical carrier or excipient and can additionally include other medicinal agents, carriers, adjuvants, and the like. Preferably, the formulation will be about 5% to 75% by weight of a composition of the invention, with the remainder consisting of suitable pharmaceutical excipients. Appropriate excipients can be tailored to the particular composition and route of administration by methods well known in the art (see, e.g., REMINGTON's PHARMACEUTICAL SCIENCES, 18TH ED., Mack Publishing Co., Easton, Pa. (1990)).

The formulations can take the form of solid, semi-solid, lyophilized power, or liquid dosage forms, such as, for example, tablets, pills, capsules, powders, solutions, suspensions, emulsions, suppositories, retention enemas, creams, ointments, lotions, gels, aerosols or the like. In embodiments where the pharmaceutical composition takes the form of a pill, tablet or capsule, the formulation can contain, along with the biologically active composition, any of the following: a diluent such as lactose, sucrose, dicalcium phosphate, and the like; a distintegrant such as starch or derivatives thereof; a lubricant such as magnesium stearate and the like; and a binder such as starch, gum acacia, polyvinylpyrrolidone, gelatin, cellulose and derivatives thereof. Compositions can be presented in unit-dose or multi-dose sealed containers, such as ampoules or vials. Doses administered to a patient should be sufficient to achieve a beneficial therapeutic response in the patient over time. The invention specifically excludes antibody fragments which do not have biological activity other than only binding a specific antigen when the term “therapeutic” or “biologically active protein” is employed. Since antigens suitable for immunization have other biological activities such as mounting an immune response, these remain included in the appropriate aspects of this invention, however. Moreover, agents that have a biological activity or a therapeutic effect by binding a specific antigen, thereby blocking ligand binding or altering the conformation of the antigen are included in this invention.

In some embodiments, a sustained-release or controlled-release formulation can be administered to an organism or to cells in culture and can carry the desired compositions. The sustained-release composition can be administered to the tissue of an organism, for example, by injection. By “sustained-release”, it is meant that the composition, preferably one encoding a transgene of interest or a biological or therapeutic agent, is made available for uptake by surrounding tissue or cells in culture for a period of time longer than would be achieved by administration of the composition in a less viscous medium, for example, a saline solution.

The compositions, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like. For delivery by inhalation, the compositions can also be delivered as dry powder (e.g., Nektar Therapeutics, San Carlos, Calif.).

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Other methods of administration include, but are not limited to, administration using angioplastic balloons, catheters, and gel formations. Methods for angioplastic balloon, catheter and gel formation delivery are well known in the art.

Imaging Methods

One of skill in the art will understand that the compositions of the present invention can by tailored for a variety of imaging uses. In one embodiment, virtual colonoscopy can be performed using the component-based system for imaging. At present, virtual colonoscopy involves essentially infusing contrast into a colon and visualizing the images on CT, then reconstructing a 3-D image. Similar techniques could be employed for MR. However, feces, mucous, and air all serve as contrast barriers and can give an artificial surface to the colon wall reconstruction. Addition of a cellular-targeting contrast would help overcome these barriers to provide a true wall reconstruction and help avoid both false-positives and false-negatives. There are several ways that the component-based system could be applied here. Most simply, the cationic efficiency backbone could be applied with a single contrast agent, for example CT, MR, or optical. Thus, the cellular surface layer could be visualized and any irregularities or obstructions detailed in the image reconstruction. However, the component based system offers the additional option of adding a specific second agent. This agent could consist of a cationic efficiency backbone, a different imaging moiety, and targeting components, for example targeting two antigens characteristic of colon cancer. The imaging moieties from the simple to the diagnostic could be selected so that one was CT contrast and the other MR contrast, or so that both were MR contrast with one being a T2 agent and the other a T1 agent. In this manner, the surface could be reconstructed as before, and any regions specific for a tumor antigen could be visualized and overlaid on the original reconstruction. Additionally, therapeutic agents could be incorporated into the targeted diagnostic system as well. Similar strategies could be applied to regional enteritis and ulcerative colitis (and again combined with therapy). Alternately, optical imaging moieties and detection methods could be employed, for example, in the case of melanoma diagnosis or management, preferably in conjunction with a fluorescent imaging moiety. The optical imaging agent can be selected for example from the group including Cy3, Cy3.5, Cy5, Cy5.5, Cy7, Cy7.5, Oregon green 488, Oregon green 500, Oregon, green 514, Green fluorescent protein, 6-FAM, Texas Red, Hex, TET, and HAMRA.

### EXAMPLES

### Example 1

This example illustrates a composition suitable for transdermal delivery of a very large complex, namely a plasmid containing the blue fluorescent protein (BFP) transgene, using a positively charged backbone or carrier of the invention.

Backbone Selection:

The positively charged backbone was assembled by covalently attaching -Gly₃Arg₇ to polylysine MW 150,000 via the carboxyl of the terminal glycine to free amines of the lysine sidechains at a degree of saturation of 18% (i.e., 18 out of each 100 lysine residues is covalently attached to a -Gly₃Arg₇). The modified backbone was designated “KNR2” to denote a second size of the peptidyl carrier. The control polycation was unmodified polylysine (designated “K2”, Sigma Chemical Co., St. Louis, Mo.) of the same size and from the same lot. An additional control polycation, Superfect® (Qiagen) which is an activated dendrimer-based agent, was selected as a reference for high in vitro transfection rates (i.e. simultaneous positive control and reference for state-of-the art efficiency versus toxicity in vitro).

Therapeutic Agent Selection:

An 8 kilobase plasmid (pSport-based template, Gibco BRL, Gaithersburg, Md.) containing the entire transgene for blue fluorescent protein (BFP) and partial flanking sequences driven by a cytomegalovirus (CMV) promoter was employed. BFP serves as an identifiable marker for cells that have been transfected, then transcribe and translate the gene and can be directly visualized (i.e. without additional staining) under fluorescence microscopy. Thus, only cells in which the complex has crossed both the plasma membrane and the nuclear membrane before payload delivery can have transgene expression. This particular plasmid has a molecular weight of approximately 2.64 million, and was thus selected to evaluate the delivery of very large therapeutics via these complexes.

Preparation of Samples:

In each case, an excess of polycation was employed to assemble a final complex that has an excess of positive charge. Although increasing charge density increases size (i.e. more backbones present per complex), increase in efficiency factor density per complex can offset these changes. Thus, an optimal may occur at low ratios (i.e. size-based) or at high ratios (i.e. density of efficiency-factor based) and both are evaluated here for KNR2. Optimal ratios for K2 efficiency and Superfect efficiency were selected based on manufacturers recommendation and prior reports on maximal efficiency. Nucleic acid-therapeutic dose was standardized across all groups as was total volume and final pH of the composition to be evaluated in cell culture.

The following mixtures were prepared:
1) K2 at a 4:1 charge ratio to a 0.5 mg/mL solution of a plasmid expressing blue fluorescent protein driven by a CMV promoter.
2) KNR2 at a ratio of 15:1 to a 0.5 mg/mL solution of a plasmid expressing blue fluorescent protein driven by a CMV promoter.
3) KNR2 at a ratio of 10:1 to a 0.5 mg/mL solution of a plasmid expressing blue fluorescent protein driven by a CMV promoter.
4) KNR2 at a ratio of 4:1 to a 0.5 mg/mL solution of a plasmid expressing blue fluorescent protein driven by a CMV promoter.
5) KNR2 at a ratio of 1.25:1 to a 0.5 mg/mL solution of a plasmid expressing blue fluorescent protein driven by a CMV promoter.
6) Superfect according to the manufacturer's recommendation at a 5:1 charge ratio to a 0.5 mg/mL solution of a plasmid expressing blue fluorescent protein driven by a CMV promoter.

Cell Culture Protocols:

All cell culture experiments were performed by observers blinded to the identity of treatment groups. On a 6-well plate, 1.0 mL of each solution was added to 70% confluent HA-VSMC primary human aortic smooth muscle cells (passage 21; ATCC, Rockville, Md.) and grown in M-199 with 10% serum for 48 hours at 37 degrees Celsius and 10% CO₂. Untreated control wells were evaluated as well and each group was evaluated at n=5 wells per group.

Analysis of Efficiency:

Low magnification photographs (10× total) of intact cell plates were obtained by blinded observers at 60 degrees, 180 degrees and 200 degrees from the top of each well using a Nikon E600 epi-fluorescence microscope with a BFP filter and plan apochromat lenses. Image Pro Plus 3.0 image analysis suite (Media Cybernetics, Silver Spring, Md.) was employed to determine the percent of total cell area that was positive. This result was normalized to total cell area for each, and reported as efficiency of gene delivery (% of total cells expressing transgene at detectible levels).

Analysis of Toxicity:

Wells were subsequently evaluated by blinded observers in a dye exclusion assay (viable cells exclude dye, while nonviable ones cannot), followed by solubilization in 0.4% SDS in phosphate buffered saline. Samples were evaluated in a Spectronic Genesys 5 UV/VIS spectrophotometer at 595 nm wavelength (blue) to quantitatively evaluate nonviable cells as a direct measure of transfection agent toxicity. Samples were standardized to identical cell numbers by adjusting concentrations to matching OD280 values prior to the OD595 measurements.

Data Handling and Statistical Analysis:

Total positive staining was determined by blinded observer via batch image analysis using Image Pro Plus software (Media Cybernetics, Silver Spring, Md.) and was normalized to total cross-sectional area to determine percent positive staining for each. Mean and standard error were subsequently determined for each group with analysis of significance at 95% confidence in one way ANOVA repeated measures using Statview software (Abacus, Berkeley, Calif.).

Results:

Efficiencies:
Results for efficiencies are as follows (mean±Standard Error):
1) 0.163±0.106%
2) 10.642±2.195%
3) 8.797±3.839%
4) 15.035±1.098%
5) 17.574±6.807%
6) 1.199±0.573%
Runs #4 and #5 exhibit statistically significant (P<0.05 by one factor ANOVA repeated measures with Fisher PLSD and TUKEY-A posthoc testing) enhancement of gene delivery efficiency relative to both polylysine alone and Superfect.

Toxicities:

Mean toxicity data are as follows (reported in AU at OD595; low values, such as present with saline alone correlate with low toxicity, while higher values, such as present in condition 1 indicate a high cellular toxicity):
Saline—0.057 A;
1) 3.460 A;
2) 0.251 A;
3) 0.291 A;
4) 0.243 A;
5) 0.297 A;
6) 0.337 A.

Conclusions:

A less toxic, more efficient gene delivery can be accomplished with a ratio of 1.25 to 4.0 of KNR2 to DNA than controls, even those of the current gold standard Superfect. This experiment confirms the capability to deliver quite large therapeutic complexes across membranes using this carrier.

### Example 2

This example illustrates the transport of a large nucleic acid across skin by a carrier of the invention after a single administration.

Backbone Selection:

The positively charged backbone was assembled by covalently attaching -Gly₃Arg₇ to polylysine MW 150,000 via the carboxyl of the terminal glycine to free amines of the lysine sidechains at a degree of saturation of 18% (i.e., 18 out of each 100 lysine residues is covalently attached to a -Gly₃Arg₇). The modified backbone was designated “KNR2” as before. The control polycation was unmodified polylysine (designated “K2”, Sigma Chemical Co., St. Louis, Mo.) of the same size and from the same lot. An additional control polycation, Superfect (Qiagen) which is an activated dendrimer-based agent, was selected as a reference for high transfection rates (i.e. simultaneous positive control and reference for state-of-the art efficiency versus toxicity in vitro).

Therapeutic Agent Selection:

For the present experiment, an 8.5 kilobase plasmid (pSport-based template, Gibco BRL, Gaithersburg, Md.) containing the entire transgene for E. Coli beta-galactosidase (βgal) and partial flanking sequences driven by a cytomegalovirus (CMV) promoter was employed. Here βgal serves as an identifiable marker for cells which have been transfected, then transcribe and translate the gene and can be directly visualized after specific staining for the foreign enzyme. Thus, only cells in which the complex has crossed skin then reached the target cell and translocated across both the plasma membrane and the nuclear membrane before payload delivery can have transgene expression. This particular plasmid has a molecular weight of approximately 2,805,000.

Preparation of Samples:

In each case, an excess of polycation is employed to assemble a final complex that has an excess of positive charge. Optimal ratios for K2 efficiency, KNR2 efficiency and Superfect efficiency were selected based on manufacturer's recommendation and prior in vitro experiments to determine maximal efficiency. Nucleic acid-therapeutic dose was standardized across all groups as was total volume and final pH of the composition to be applied topically. Samples were prepared as follows:
Group labeled AK1: 8 micrograms of βgal plasmid (p/CMV-sport-βgal) per final aliquot (i.e. 80 micrograms total) and peptidyl carrier KNR2 at a charge ratio of 4:1 were mixed to homogeneity and diluted to 200 microliters with phosphate buffered saline. The resulting composition was mixed to homogeneity with 1.8 ml of Cetaphil moisturizer and aliquoted in 200 microliter portions for in vivo experiments.
Group labeled AL1: 8 micrograms of βgal plasmid (p/CMV-sport-βgal) per final aliquot (i.e. 80 micrograms total) and K2 at a charge ratio of 4:1 were mixed to homogeneity and diluted to 200 microliters with phosphate buffered saline. The resulting composition was mixed to homogeneity with 1.8 ml of Cetaphil and aliquoted in 200 microliter portions for in vivo experiments.
Group labeled AM1: 8 micrograms of βgal plasmid (p/CMV-sport-βgal) per final aliquot (i.e. 80 micrograms total) and Superfect at a charge ratio of 5:1 were mixed to homogeneity and diluted to 200 microliters with phosphate buffered saline. The resulting composition was mixed to homogeneity with 1.8 ml of Cetaphil and aliquoted in 200 microliter portions for in vivo experiments.

Animal experiments to Determine Transdermal Delivery Efficiencies After Single Treatment with Peptidyl Carriers and Nucleic Acid Therapeutics:

Animals were anesthetized via inhalation of isoflurane during application of treatments. After being anesthetized, C57 black 6 mice (n=4 per group) had metered 200 microliter doses of the appropriate treatment applied to the cranial portion of dorsal back skin (selected because the mouse cannot reach this region with mouth or limbs). Animals did not undergo depilatory treatment. Animals were recovered in a controlled heat environment to prevent hypothermia and once responsive were provided food and water ad libitum overnight. Twenty-four hours post-treatment, mice were euthanized via inhalation of CO₂, and treated skin segments were harvested at full thickness by blinded observers. Treated segments were divided into three equal portions the cranial portion was fixed in 10% neutral buffered formalin for 12-16 hours then stored in 70% ethanol until paraffin embedding. The central portion was snap-frozen and employed directly for beta-galactosidase staining at 37 degrees Celsius on sections as previously described (Waugh, J. M., M. Kattash, J. Li, E. Yuksel, M. D. Kuo, M. Lussier, A. B. Weinfeld, R. Saxena, E. D. Rabinovsky, S. Thung, S. L. C. Woo, and S. M. Shenaq. Local Overexpression of Tissue Plasminogen Activator to Prevent Arterial Thrombosis in an in vivo Rabbit Model. Proc Natl Acad Sci USA. 1999 96(3): 1065-1070. Also: Elkins C J, Waugh J M, Amabile P G, Minamiguchi H, Uy M, Sugimoto K, Do Y S, Ganaha F, Razavi M K, Dake M D. Development of a platform to evaluate and limit in-stent restenosis. Tissue Engineering June 2002;8(3): 395-407). The treated caudal segment was snap frozen for solubilization studies.

Toxicity:

Toxicity was evaluated by dye exclusion on paired sections to those analyzed for efficiency above. Sections only underwent staining for either efficiency or for toxicity since the methods are not reliably co-employed. For toxicity analyses, the sections were immersed in exclusion dye for 5 minutes, then incubated at 37 degrees Celsius for 30 minutes at 10% CO₂. Any cells that did not exclude the dye in this period of time were considered non-viable.

Data Handling and Statistical Analyses:

Data collection and image analysis were performed by blinded observers. Sections stained as above were photographed in their entirety on a Nikon E600 microscope with plan-apochromat lenses. Resulting images underwent batch image analysis processing using Image Pro Plus software as before with manual confirmation to determine number positive for beta-galactosidase enzyme activity (blue with the substrate method employed here) or cellular toxicity. These results were normalized to total cross-sectional number of cells by nuclear fast red staining for each and tabulated as percent cross-sectional positive staining. Subsequently, mean and standard error were subsequently determined for each group with analysis of significance at 95% confidence in one way ANOVA repeated measures using Statview software (Abacus, Berkeley, Calif.).

Results:

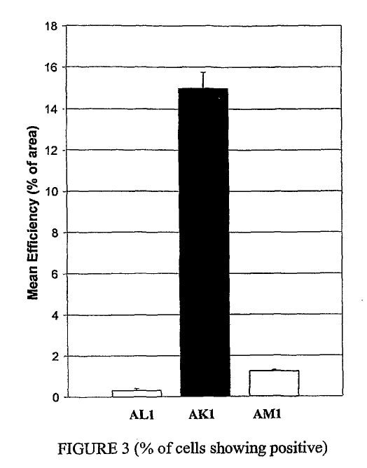

Results are summarized in the table below and illustrated in FIG. 3. The positively charged peptidyl transdermal delivery carrier achieved statistically significant increases in delivery efficiency and transgene expression versus both K2 (negative control essentially) and the benchmark standard for efficiency, Superfect. While Superfect did achieve statistically significant improvements over K2, KNR2 had greater than an order of magnitude improvement in delivery efficiency versus Superfect in this model system.

### Example 2

Mean and standard error for beta-galactosidase positive cells as percent of total number by treatment group.

| | | Std. |
|---|---|---|
| Group | Mean | Error. |
| AK1 | 15.00 | 0.75 |
| AL1 | 0.03 | 0.01 |
| AM1 | 1.24 | 0.05 |

P = 0.0001 (Significant at 99%)

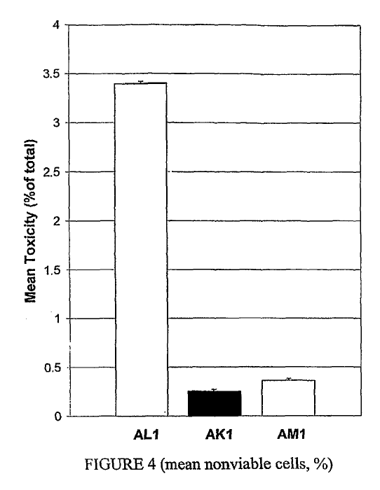

Results for toxicity are presented in FIG. 4, which depicts the percent of total area that remained nonviable 24 hours post treatment. Here, K2 exhibits statistically significant cellular toxicity relative to KNR2 or Superfect, even at a dose where K2 has low efficiency of transfer as described previously (Amabile, P. G., J. M. Waugh, T. Lewis, C. J. Elkins, T. Janus, M. D. Kuo, and M. D. Dake. Intravascular Ultrasound Enhances in vivo Vascular Gene Delivery. J. Am. Col. Cardiol. June2001; 37(7):1975-80).

Conclusion:

The peptidyl transdermal carrier can transport large complexes across skin with high efficiencies, particularly given the constraints of transgene expression and total complex size discussed previously. Positive area here, rather than positive number was employed for analyses since (1) the method is greatly simplified and has greater accuracy in image analysis, (2) point demonstrations of efficiencies had already been afforded in II.B conclusively, (3) area measurements provide a broader scope for understanding in vivo results since noncellular components occupy a substantial portion of the cross section, and (4) comparison to still larger nonpeptidyl carrier complexes was facilitated

### Example 3

This example illustrates the transdermal delivery of a large nucleic acid-based therapeutic across skin using a positively charged peptidyl carrier of the invention in seven sequential daily applications.

Backbone Selection:

The positively charged peptidyl backbone was assembled by covalently attaching -Gly₃Arg₇ to polylysine MW 150,000 via the carboxyl of the terminal glycine to free amines of the lysine sidechains at a degree of saturation of 18% (i.e., 18 out of each 100 lysine residues is covalently attached to a -Gly₃Arg₇). The modified backbone was designated “KNR2”. The control polycation was unmodified polylysine (designated “K2”, Sigma Chemical Co., St. Louis, Mo.) of the same size and from the same lot.

Therapeutic Agent Selection:

For the present experiment, an 8.5 kilobase plasmid (pSport-based template, Gibco BRL, Gaithersburg, Md.) containing the entire transgene for E. Coli beta-galactosidase (βgal) and partial flanking sequences driven by a cytomegalovirus (CMV) promoter was employed. This particular plasmid has a molecular weight of approximately 2,805,000 and was thus selected to evaluate delivery of very large therapeutics across skin via the peptidyl carriers.

Preparation of Samples:

In each case, an excess of polycation was employed to assemble a final complex that has an excess of positive charge. Experimental ratios were selected to parallel the single dose experiments presented in the previous experiment. Nucleic acid-therapeutic dose was standardized across all groups as was total volume and final pH of the composition to be applied topically. Samples were prepared as follows:
Group labeled AK1: 8 micrograms of βgal plasmid (p/CMV-sport-βgal) per final aliquot (i.e. 240 micrograms total) and peptidyl carrier KNR2 at a charge ratio of 4:1 were mixed to homogeneity and diluted to 600 microliters with phosphate buffered saline. The resulting composition was mixed to homogeneity with 5.4 ml of Cetaphil and aliquoted in 200 microliter portions for in vivo experiments.
Group labeled AL1: 8 micrograms of βgal plasmid (p/CMV-sport-βgal) per final aliquot (i.e. 240 micrograms total) and K2 at a charge ratio of 4:1 were mixed to homogeneity and diluted to 600 microliters with phosphate buffered saline. The resulting composition was mixed to homogeneity with 5.4 ml of Cetaphil and aliquoted in 200 microliter portions for in vivo experiments.

Animal Experiments to Determine Cumulative Transdermal Delivery Efficiencies After 7 Once-Daily Treatments with Peptidyl Carriers and Nucleic Acid Therapeutics:

Animals were anesthetized via inhalation of isoflurane during application of treatments. After being anesthetized, C57 black 6 mice (n=4 per group) had metered 200 microliter doses of the appropriate treatment applied to the cranial portion of dorsal back skin (selected because the mouse cannot reach this region with mouth or limbs). Animals did not undergo depilatory treatment. Animals were recovered in a controlled heat environment to prevent hypothermia and once responsive were provided food and water ad libitum overnight. This procedure was repeated once daily at the same approximate time of day for 7 days. After 7 days treatment, mice were euthanized via inhalation of CO₂, and treated skin segments were harvested at full thickness by blinded observers. Treated segments were divided into three equal portions the cranial portion was fixed in 10% neutral buffered formalin for 12-16 hours then stored in 70% ethanol until paraffin embedding. The central portion was snap-frozen and employed directly for beta-galactosidase staining at 37 degrees Celsius on sections as previously described. The treated caudal segment was snap frozen for solubilization studies.

Data Handling and Statistical Analyses:

Data collection and image analysis were performed by blinded observers. Sections stained as above were photographed in their entirety on a Nikon E600 microscope with plan-apochromat lenses. Resulting images underwent batch image analysis processing using Image Pro Plus software as before with manual confirmation to determine area positive for beta-galactosidase enzyme activity. These results were normalized to total cross-sectional area for each and tabulated as percent cross-sectional positive staining. Subsequently, mean and standard error were subsequently determined for each group with analysis of significance at 95% confidence in one way ANOVA repeated measures using Statview 'software (Abacus, Berkeley, Calif.).

Results:

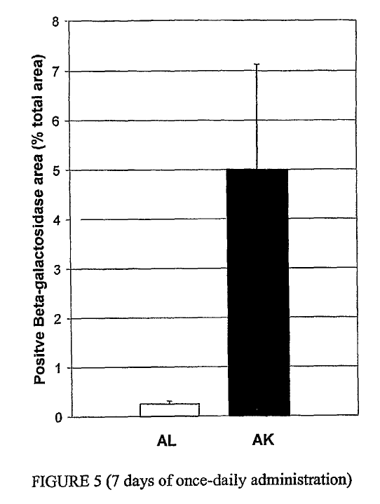

Results are summarized in the table below and illustrated in FIG. 5. The peptidyl transdermal delivery carrier achieved statistically significant increases in delivery efficiency and transgene expression versus K2.

### Example 3

Mean and standard error for cumulative transgene expression of beta-galactosidase as percent of total area after 7 once-daily applications for each treatment group.

| Group | Mean | Std. Error. |
|---|---|---|
| AK | 5.004 | 2.120 |
| AL | 0.250 | 0.060 |

P = 0.0012 (Significant at 99%)

### Example 4

### (Non-Peptidyl Carrier)

This example illustrates the transdermal delivery of a large nucleic acid-based therapeutic across skin, using a positively charged non-peptidyl carrier of the invention in seven sequential daily applications.

Backbone Selection:

The positively charged backbone was assembled by covalently attaching -Gly₃Arg₇ to polyethyleneimine (PEI) MW 1,000,000 via the carboxyl of the terminal glycine to free amines of the PEI sidechains at a degree of saturation of 30% (i.e., 30 out of each 100 lysine residues is covalently attached to a -Gly₃Arg₇). The modified backbone was designated “PEIR” to denote the large nonpeptidyl carrier. The control polycation was unmodified PEI (designated “PEI”, Sigma Chemical Co., St. Louis, Mo.) of the same size and from the same lot.

Therapeutic Agent Selection:

For the present experiment, an 8.5 kilobase plasmid (pSport-based template, Gibco BRL, Gaithersburg, Md.) containing the entire transgene for E. Coli beta-galactosidase (βgal) and partial flanking sequences driven by a cytomegalovirus (CMV) promoter was employed. This particular plasmid has a molecular weight of approximately 2,805,000.

Preparation of Samples:

In each case, an excess of polycation was employed to assemble a final complex that has an excess of positive charge. Nucleic acid-therapeutic dose was standardized across all groups as was total volume and final pH of the composition to be applied topically. Samples were prepared as follows:
Group labeled AS: 8 micrograms of βgal plasmid (p/CMV-sport-βgal) per final aliquot (i.e. 240 micrograms total) and control PEI at a charge ratio of 5:1 were mixed to homogeneity and diluted to 600 microliters with Tris-EDTA buffer. The resulting composition was mixed to homogeneity with 5.4 ml of Cetaphil and aliquoted in 200 microliter portions for in vivo experiments.
Group labeled AT: 8 micrograms of βgal plasmid (p/CMV-sport-βgal) per final aliquot (i.e. 240 micrograms total) and composite nonpeptidyl carrier PEIR (“PEIR”) at a charge ratio of 5:1 were mixed to homogeneity and diluted to 600 microliters with Tris-EDTA buffer. The resulting composition was mixed to homogeneity with 5.4 ml of Cetaphil and aliquoted in 200 microliter portions for in vivo experiments.
Group labeled AU: 8 micrograms of βgal plasmid (p/CMV-sport-βgal) per final aliquot (i.e. 240 micrograms total) and highly purified Essentia nonpeptidyl carrier PEIR (“pure PEIR”) at a charge ratio of 5:1 were mixed to homogeneity and diluted to 600 microliters with Tris-EDTA buffer. The resulting composition was mixed to homogeneity with 5.4 ml of Cetaphil and aliquoted in 200 microliter portions for in vivo experiments.

Animal Experiments to Determine Cumulative Transdermal Delivery Efficiencies After 7 Once-Daily Treatments with Nonpeptidyl Carriers and Nucleic Acid Therapeutics:

Animals were anesthetized via inhalation of isoflurane during application of treatments. After being anesthetized, C57 black 6 mice (n=3 per group) had metered 200 microliter doses of the appropriate treatment applied to the cranial portion of dorsal back skin (selected because the mouse cannot reach this region with mouth or limbs). Animals did not undergo depilatory treatment. Animals were recovered in a controlled heat environment to prevent hypothermia and once responsive were provided food and water ad libitum overnight. This procedure was repeated once daily at the same approximate time of day for 7 days. After 7 days treatment, mice were euthanized via inhalation of CO₂, and treated skin segments were harvested at full thickness by blinded observers. Treated segments were divided into three equal portions the cranial portion was fixed in 10% neutral buffered formalin for 12-16 hours then stored in 70% ethanol until paraffin embedding. The central portion was snap-frozen and employed directly for beta-balactosidase staining at 37 degrees Celsius on sections as previously described. The treated caudal segment was snap frozen for solubilization studies.

Data Handling and Statistical Analyses:

Data collection and image analysis were performed by blinded observers. Sections stained as above were photographed in their entirety on a Nikon E600 microscope with plan-apochromat lenses. Resulting images underwent batch image analysis processing using Image Pro Plus software with manual confirmation to determine area positive for beta-galactosidase enzyme activity. These results were normalized to total cross-sectional area for each and tabulated as percent cross-sectional positive staining. Subsequently, mean and standard error were subsequently determined for each group with analysis of significance at 95% confidence in one way ANOVA repeated measures using Statview software (Abacus, Berkeley, Calif.).

Results:

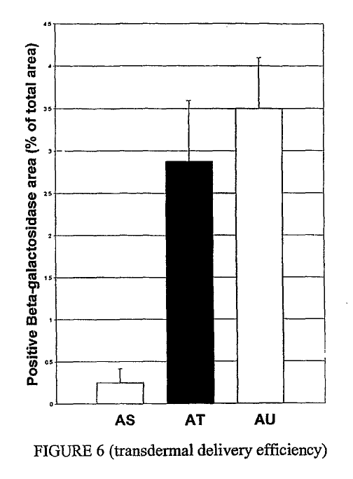

Results are summarized in the table below and illustrated in FIG. 6. The nonpeptidyl transdermal delivery carrier—in both a composite form and in an ultrapure form—achieved statistically significant increases in delivery efficiency and transgene expression versus PEI. The ultrapure form of PEIR exhibited trending toward higher efficiencies than standard PEIR consistent with the higher calculated specific activity of the reagent.

### Example 4

Mean and standard error for cumulative transgene expression of beta-galactosidase as percent of total area after 7 once daily applications for each treatment group.

| Group | Mean | Std. Error. |
|---|---|---|
| AS | 0.250 | 0.164 |
| AT | 2.875 | 0.718 |
| AU | 3.500 | 0.598 |

P = 0.0058 (Significant at 99%)

Conclusions:

The nonpeptidyl transdermal carrier can transport large complexes across skin with high efficiencies, particularly given the constraints of transgene expression and total complex size discussed previously. While the efficiencies are not as great as those obtained with the smaller complexes of the peptidyl carriers), significant gains were accomplished. Of note, the distribution of transgene expression using the large nonpeptidyl complexes was almost exclusively hair follicle-based, while the results for the peptidyl carriers were diffuse throughout the cross-sections. Thus, size and backbone tropism can be employed for a nano-mechanical targeting of delivery.

### Example 5

This experiment demonstrates the use of a peptidyl carrier to transport a large complex containing an intact labeled protein botulinum toxin across intact skin after a single time administration relative to controls.

Backbone Selection:

The positively charged backbone was assembled by covalently attaching -Gly₃Arg₇ to polylysine MW 112,000 via the carboxyl of the terminal glycine to free amines of the lysine side chains at a degree of saturation of 18% (i.e., 18 out of each 100 lysine residues is covalently attached to a -Gly₃Arg₇). The modified backbone was designated “KNR”. The control polycation was unmodified polylysine (designated “K”, Sigma Chemical Co., St. Louis, Mo.) of the same size and from the same lot.

Therapeutic Agent:

Botox® brand of botulinum toxin A (Allergan) was selected for this experiment. It has a molecular weight of approximately 150,000.

Preparation of Samples:

The botulinum toxin was reconstituted according to the manufacturer's instructions. An aliquot of the protein was biotinylated with a calculated 12-fold molar excess of sulfo-NHS-LC biotin (Pierce Chemical). The labeled product was designated “Btox-b”.

In each case, an excess of polycation was employed to assemble a final complex that has an excess of positive charge as in delivery of highly negative large nucleic acid complexes. A net neutral or positive charge prevents repulsion of the protein complex from highly negative cell surface proteoglycans and extracellular matrix. Btox-b dose was standardized across all groups, as was total volume and final pH of the composition to be applied topically. Samples were prepared as follows:
Group labeled “JMW-7”: 2.0 units of Btox-b per aliquot (i.e. 20 U total) and peptidyl carrier KNR at a calculated MW ratio of 4:1 were mixed to homogeneity and diluted to 200 microliters with phosphate buffered saline. The resulting composition was mixed to homogeneity with 1.8 ml of Cetaphil and aliquoted in 200 microliter portions.
Group labeled “JMW-8”: 2.0 units of Btox-b per aliquot (i.e. 20 U total) and K at a charge ratio of 4:1 were mixed to homogeneity and diluted to 200 microliters with phosphate buffered saline. The resulting composition was mixed to homogeneity with 1.8 ml of Cetaphil and aliquoted in 200 microliter portions.

Animal Experiments to Determine Transdermal Delivery Efficiencies After Single Time Treatment with Peptidyl Carriers and Labeled Botulinum Toxin:

Animals were anesthetized via inhalation of isoflurane during application of treatments. After being anesthetized, C57 black 6 mice (n=4 per group) underwent topical application of metered 200 microliter dose of the appropriate treatment applied to the cranial portion of dorsal back skin (selected because the mouse cannot reach this region with mouth or limbs). Animals did not undergo depilation. At 30 minutes after the initial treatment, mice were euthanized via inhalation of CO₂, and treated skin segments were harvested at full thickness by blinded observers. Treated segments were divided into three equal portions; the cranial portion was fixed in 10% neutral buffered formalin for 12-16 hours then stored in 70% ethanol until paraffin embedding. The central portion was snap-frozen and employed directly for biotin visualization by blinded observers as summarized below. The treated caudal segment was snap frozen for solubilization studies.

Biotin visualization was conducted as follows. Briefly, each section was immersed for 1 hour in NeutrAvidin® buffer solution. To visualize alkaline phosphatase activity, cross sections were washed in saline four times then immersed in NBT/BCIP (Pierce Scientific) for 1 hour. Sections were then rinsed in saline and photographed in entirety on a Nikon E600 microscope with plan-apochromat lenses.

Data Handling and Statistical Analysis:

Total positive staining was determined by blinded observer via batch image analysis using Image Pro Plus software (Media Cybernetics, Silver Spring, Md.) and was normalized to total cross-sectional area to determine percent positive staining for each. Mean and standard error were subsequently determined for each group with analysis of significance at 95% confidence in one way ANOVA repeated measures using Statview software (Abacus, Berkeley, Calif.).

Results:

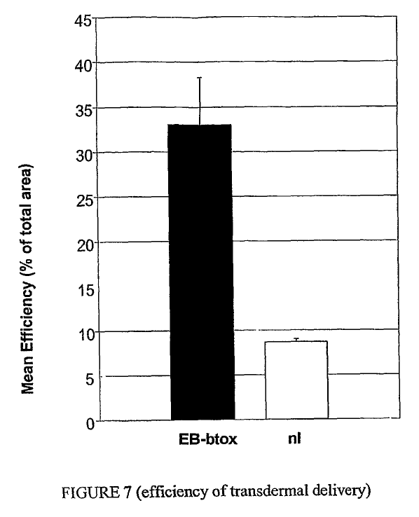

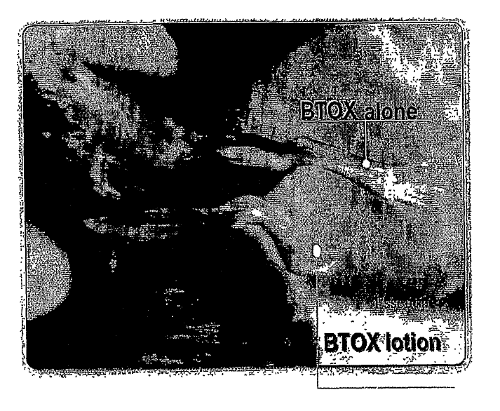

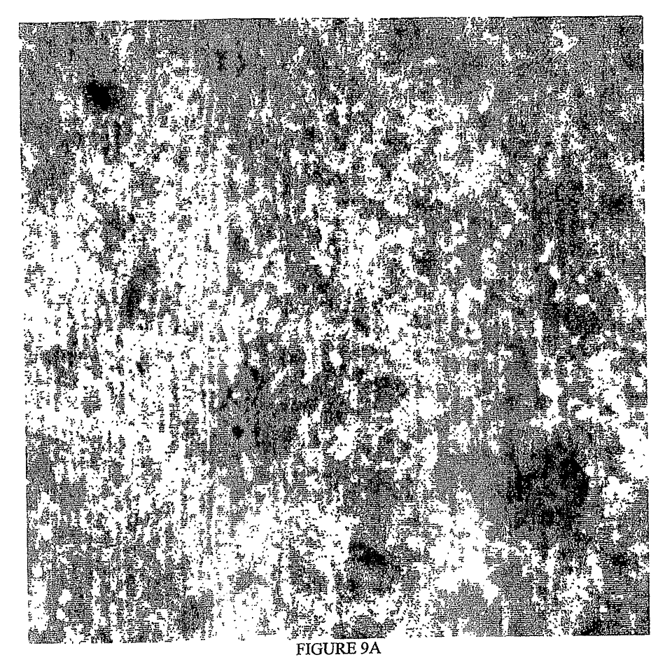

The mean cross-sectional area positive for biotinylated botulinum toxin was reported as percent of total area after single-time topical administration of Btox-b with either KNR (“EB-Btox”) or K (“n1”). The results are presented in the following table and are illustrated in FIG. 7. In FIG. 7, the area positive for label was determined as percent of total area after three days of once daily treatment with “EB-Btox” which contained Btox-b and the peptidyl carrier KNR and “n1”, which contained Btox-b with polycation K as a control. Mean and standard error are depicted for each group.

### Example 5

Mean and standard error for labeled botulinum toxin area as percent of total cross-section after single time topical administration of Btox-b with KNR (JMW-7) or K (JMW-8) for 30 minutes.

| Group | Mean | Std. Error |
|---|---|---|
| JMW-7 | 33.000 | 5.334 |
| JMW-8 | 8.667 | 0.334 |

P = 0.0001 (Significant at 99%)

### Example 6

Example 5 demonstrated that the peptidyl transdermal carrier allowed efficient transfer of botulinum toxin after topical administration in a murine model of intact skin. However, this experiment did not indicate whether the complex protein botulinum toxin was released in a functional form after translocation across skin. The following experiment was thus constructed to evaluate whether botulinum toxin can be therapeutically delivered across intact skin as a topical agent using this peptidyl carrier (again, without covalent modification of the protein).

The positively charged backbone was again assembled by covalently attaching -Gly₃Arg₇ to polylysine MW 112,000 via the carboxyl of the terminal glycine to free amines of the lysine side chains at a degree of saturation of 18% (i.e., 18 out of each 100 lysine residues is covalently attached to a -Gly₃Arg₇). The modified backbone was designated “KNR”. Control polycation was unmodified polylysine (designated “K”, Sigma Chemical Co., St. Louis, Mo.) of the same size and from the same lot. The same botulinum toxin therapeutic agent was used as in Example 5, and was prepared in the same manner. Samples were prepared as follows:
Group labeled “JMW-9”: 2.0 units of botulinum toxin per aliquot (i.e. 60 U total) and peptidyl carrier KNR at a calculated MW ratio of 4:1 were mixed to homogeneity and diluted to 600 microliters with phosphate buffered saline. The resulting composition was mixed to homogeneity with 5.4 ml of Cetaphil and aliquoted in 200 microliter portions.
Group labeled “JMW-10”: 2.0 units of botulinum toxin per aliquot (i.e. 60 U total) and K at a charge ratio of 4:1 were mixed to homogeneity and diluted to 600 microliters with phosphate buffered saline. The resulting composition was mixed to homogeneity with 5.4 ml of Cetaphil and aliquoted in 200 microliter portions.
Group labeled “JMW-11”: 2.0 units of botulinum toxin per aliquot (i.e. 60 U total) without polycation was diluted to 600 microliters with phosphate buffered saline. The resulting composition was mixed to homogeneity with 5.4 ml of Cetaphil and aliquoted in 200 microliter portions.

Animal Experiments to Determine Therapeutic Efficacy After Single Time Treatment with Peptidyl Carriers and Botulinum Toxin:

Animals were anesthetized via inhalation of isoflurane during application of treatments. After being anesthetized, C57 black 6 mice (n=4 per group) underwent topical application of metered 400 microliter dose of the appropriate treatment applied uniformly from the toes to the mid-thigh. Both limbs were treated, and treatments were randomized to either side. Animals did not undergo depilation. At 30 minutes after the initial treatment, mice were evaluated for digital abduction capability according to published digital abduction scores for foot mobility after botulinum toxin administration (Aoki, K R. A comparison of the safety margins of botulinum neurotoxin serotypes A, B, and F in mice. Toxicon. December 2001; 39(12): 1815-20). Mouse mobility was also subjectively assessed.

Data Handling and Statistical Analysis:

Digital abduction scores were tabulated independently by two blinded observers. Mean and standard error were subsequently determined for each group with analysis of significance at 95% confidence in one way ANOVA repeated measures using Statview software (Abacus, Berkeley, Calif.).

Results:

Mean digital abduction scores after single-time topical administration of botulinum toxin with KNR (“JMW-9”), K (“JMW-10”) or diluent without polycation (“JMW-11”), are presented in the table below and illustrated in the representative photomicrograph of FIG. 8. The peptidyl carrier KNR afforded statistically significant functional delivery of the botulinum toxin across skin relative to both controls, which were comparable to one another. Additional independent repetitions (total of three independent experiments all with identical conclusions in statistically significant paralysis from topical botulinum toxin with KNR but not controls) of the present experiment confirmed the present findings and revealed no significant differences between topical botulinum toxin with or without K (i.e. both controls). Interestingly, the mice consistently ambulated toward a paralyzed limb (which occurred in 100% of treated animals and 0% of controls from either control group). As shown in FIG. 8, a limb treated with botulinum toxin plus the control polycation polylysine or with botulinum toxin without polycation (“Btox alone”) can mobilize digits (as a defense mechanism when picked up), but the limbs treated with botulinum toxin plus the peptidyl carrier KNR (“Essentia Btox lotion”) could not be moved.

### Example 6

Digital abduction scores 30 minutes after single-time topical application of botulinum toxin with the peptidyl carrier KNR (“JMW-9”), with a control polycation K (“JMW-10”), or alone (“JMW-11”).

| Group | Mean | Std. Error |
|---|---|---|
| JMW-9 | 3.333 | 0.333 |
| JMW-10 | 0.333 | 0.333 |
| JMW-11 | 0.793 | 0.300 |

P = 0.0351 (Significant at 95%)

Conclusions:

This experiment serves to demonstrate that the peptidyl transdermal carrier can transport a therapeutically effective amount of botulinum therapeutic across skin without covalent modification of the therapeutic. The experiment also confirms that botulinum toxin does not function when applied topically in controls.

### Example 7

This experiment demonstrates the performance of a non-peptidyl carrier in the invention.

Backbone Selection:

The positively charged backbone was assembled by covalently attaching -Gly₃Arg₇ to polyethyleneimine (PEI) MW 1,000,000 via the carboxyl of the terminal glycine to free amines of the PEI side chains at a degree of saturation of 30% (i.e., 30 out of each 100 lysine residues is covalently attached to a -Gly₃Arg₇). The modified backbone was designated “PEIR” to denote the large nonpeptidyl carrier. Control polycation was unmodified PEI (designated “PEI”, Sigma Chemical Co., St. Louis, Mo.) of the same size and from the same lot. The same botulinum toxin therapeutic agent was used as in example 5.

Botulinum toxin was reconstituted from the BOTOX® product according to the manufacturer's instructions. In each case, an excess of polycation was employed to assemble a final complex that had an excess of positive charge as in delivery of highly negative large nucleic acid complexes. A net neutral or positive charge prevents repulsion of the protein complex from highly negative cell surface proteoglycans and extracellular matrix. The botulinum toxin dose was standardized across all groups as was total volume and final pH of the composition to be applied topically. Samples were prepared as follows:
Group labeled “AZ”: 2.0 units of botulinum toxin per aliquot (i.e. 60 U total) and the nonpeptidyl carrier PEIR in ultrapure form at a calculated MW ratio of 5:1 were mixed to homogeneity and diluted to 600 microliters with phosphate buffered saline. The resulting composition was mixed to homogeneity with 5.4 ml of Cetaphil and aliquoted in 200 microliter portions.
Group labeled “BA”: 2.0 units of botulinum toxin per aliquot (i.e. 60 U total) and PEI at a charge ratio of 5:1 were mixed to homogeneity and diluted to 600 microliters with phosphate buffered saline. The resulting composition was mixed to homogeneity with 5.4 ml of Cetaphil and aliquoted in 200 microliter portions.

Animal Experiments to Determine Therapeutic Efficacy After Single Time Treatment:

Animals were anesthetized via inhalation of isoflurane during application of treatments. After being anesthetized, C57 black 6 mice (n=3 per group) underwent topical application of metered 400 microliter dose of the appropriate treatment applied uniformly from the toes to the mid-thigh. Both limbs were treated, and treatments were randomized to either side. Animals did not undergo depilation. At 30 minutes after the initial treatment, mice were evaluated for digital abduction capability according to published digital abduction scores for foot mobility after botulinum toxin administration (Aoki, K R. A comparison of the safety margins of botulinum neurotoxin serotypes A, B, and F in mice. Toxicon. December 2001; 39(12): 1815-20). Mouse mobility was also subjectively assessed.

Data Handling and Statistical Analysis:

Digital abduction scores were tabulated independently by two blinded observers. Mean and standard error were subsequently determined for each group with analysis of significance at 95% confidence in one way ANOVA repeated measures using Statview software (Abacus, Berkeley, Calif.).

Results:

Mean digital abduction scores after single-time topical administration of botulinum toxin with ultrapure PEIR (“AZ”), or control polycation PEI (“BA”), and repetition (single independent repetition for this experiment), are presented in the tables below. The nonpeptidyl carrier PEIR afforded statistically significant functional delivery of botulinum toxin across skin relative to controls. As before, animals were observed to walk in circles toward the paralyzed limbs.

### Example 7

Repetition 1. Digital abduction scores 30 minutes after single-time topical administration of Botulinum toxin with ultrapure PEIR (“AZ”), or control polycation PEI (“BA”). Mean and standard error are presented.

| Group | Mean | Std. Error |
|---|---|---|
| BA | 0.833 | 0.307 |
| AZ | 3.917 | 0.083 |

P = 0.0002 (Significant at 99%)

### Example 7

Repetition 2. Digital abduction scores 30 minutes after single-time topical administration of Botulinum toxin with ultrapure PEIR (“AZ1”), or control polycation PEI (“BA1”). Mean and standard error are presented.

| Group | Mean | Std. Error |
|---|---|---|
| BA1 | 0.333 | 0.211 |
| AZ1 | 3.833 | 0.167 |

P = 0.0001 (Significant at 99%)

Conclusions:

This experiment demonstrated that the nonpeptidyl transdermal carrier can transport therapeutic doses of botulinum toxin across skin without prior covalent modification of the botulinum toxin. These findings complement those with peptidyl transfer agents. The option of using a nonpeptidyl or a peptidyl carrier to achieve the therapeutic effect will allow tailoring to specific circumstances, environments, and methods of application and add to the breadth of the transdermal delivery platform of this invention.

In these examples botulinum toxin penetration with either peptidyl or nonpeptidyl carriers versus topical botulinum toxin without the carrier further establishes utility for transdermal penetration of antigens for immunization, particularly for immunization with antigens that cross skin poorly otherwise such as botulinum. Delivery of a functional botulinum toxin ensures that at least four distinct epitopes have been delivered transdermally in an intact state; the fact that functional botulinum toxin was not delivered in the absence of the carrier in either example confirms that the carrier affords significant immunization potential relative to the agent in the absence of the carrier. Since immunization requires that the antigens cross skin in a sufficient quantity to mount an immune response, this approach allows transdermal delivery of an antigen for immunization. Since this approach does not require covalent modification of the antigen and need not involve viral gene transfer, a number of advantages arise in terms of safety stability, and efficiency.

### Example 8

This experiment details production of peptidyl and nonpeptidyl carriers with TAT efficiency factors, as well as assembly of these carriers with botulinum toxins.

Coupling of Polyethylene Imine (PEI) to TAT Fragment GGGRKKRRQRRR:

The TAT fragment GGGRKKRRQRRR (6 mg, 0.004 mmol, Sigma Genosys, Houston, Tex.), lacking all sidechain protecting groups, was dissolved in 1 ml of 0.1M MES buffer. To this was added EDC (3 mg, 0.016 mmol) followed by PEI 400 k molecular weight 50% solution (w:v) in water, (˜0.02 ml, ˜2.5×10−5 mmol) The pH was determined to be 7.5 by test paper. Another 1 ml portion of 0.1M MES was added and the pH was adjusted to ˜5 by addition of HCl. Another portion of EDC (5 mg, 0.026 mmol) was added and the reaction, pH˜5 was stirred overnight. The next morning, the reaction mixture was frozen and lyophilized.

A column (1 cm diameter×14 cm height) of Sephadex G-25 (Amersham Biosciences Corp., Piscataway, N.J.) was slurried in sterile 1× PBS. The column was standardized by elution of FITC dextrans (Sigma, St Louis, Mo.) having 19 kD molecular weight. The standard initially eluted at 5 ml PBS, had mid peak at 6 ml and tailed at 7 ml. The lyophilized reaction mixture from above was dissolved in a small volume PBS and applied to the column. It was eluted by successive applications of 1 ml PBS. Fractions were collected with the first one consisting of the first 3 ml eluted, including the reaction volume. Subsequent fractions were 1 ml.

The fractions eluted were assayed for UV absorbance at 280 nm. Fractions 3, 4 and 5 corresponding to 5-7 ml defined a modest absorbance peak. All fractions were lyophilized and IR spectra were taken. The characteristic guanidine triple peak (2800-3000 cm−1) of the TAT fragment was seen in fractions 4-6. These fractions also showed an amide stretch at 1700 cm−1 thus confirming the conjugate of the TAT fragment and PEI.

Another iteration was run using the TAT fragment GGGRKKRRQRRR (11.6 mg, 0.007 mmol). This amount was calculated such that one in 30 of the PEI amines would be expected to be reacted with TAT fragment. This approximates the composition of the original polylysine-oligoarginine (KNR) efficiency factor described above. Successful covalent attachment of the TAT fragment to the PEI animes was confirmed by IR as above.

Coupling of Polylysine to TAT Fragment:

To a solution of polylysine (10 mg 1.1×10-4 mmol; Sigma) in 1 ml of 0.1M MES, pH ˜4.5 was added TAT fragment (4 mg, 0.003 mmol) then EDC (3.5 mg, 0.0183 mmol). The resulting reaction mixture (pH˜4.5) was stirred at RT. The reaction was frozen at −78° C. overnight. The next day the reaction mixture was thawed to RT and the pH was adjusted to ˜8 by the addition of saturated sodium bicarbonate. The reaction mixture was applied directly to a Sephadex G-25 column constituted and standardized as described above. It was eluted in seven 1 ml fractions starting after 5 ml. UV 280 absorbance was taken, revealing a relative peak in fraction 2,3 and 4. IR of the lyophilized fractions revealed the characteristic guanidine peak (2800-3000 cm−1) in fractions 1-7. Fraction 1 had a strong peak at 1730 cm−1 and nothing at 1600 cm−1, for fractions 2-6 the opposite was true. Thus, successful covalent attachment of the TAT fragment to a peptidyl carrier, polylysine, was confirmed.

The covalently attached TAT fragment and PEI (PEIT) and the covalently attached TAT fragment and polylysine (KNT) were subsequently mixed with botulinum toxin to form a noncovalent complex as below:
Group labeled “JL-1”: 2.0 units of Btox-b per aliquot (i.e. 20 U total) and PEIT at a charge ratio of 4:1 were mixed to homogeneity and diluted to 200 microliters with phosphate buffered saline.
Group labeled “JL-2”: 2.0 units of Btox-b per aliquot (i.e. 20 U total) and KNT at a charge ratio of 4:1 were mixed to homogeneity and diluted to 200 microliters with phosphate buffered saline.

After noncovalent complex formation, particles were centrifuged at 12,000×g in a rotary microcentrifuge for 5 minutes, then resuspended in 20 microliters of deionized water and evaporated on a Germanium attenuated total reflectance cell for IR. Presence of Btox-b in the complexes was thus confirmed. Overall, this experiment confirmed that synthetic schemes could be applied to other efficiency factors and the resulting carriers can be complexed with a biologically active agent—in this case botulinum toxin—as in prior examples using carriers with oligoarginine positively charged branching or efficiency groups.

### Example 9

This experiment demonstrates the performance of a peptidyl carrier for imaging of a specific antigen. In this example, complexes of one of the Essentia peptidyl carriers, KNR2, with optical imaging moieties and modified antibodies targeting melanoma are suitable for topical detection of melanoma.

Backbone Selection:

The positively charged peptidyl backbone was assembled by covalently attaching -Gly₃Arg₇ to polylysine MW 150,000 via the carboxyl of the terminal glycine to free amines of the lysine sidechains at a degree of saturation of 18% (i.e., 18 out of each 100 lysine residues is covalently attached to a -Gly₃Arg₇). The modified backbone was designated “KNR2”. The control polycation was unmodified polylysine (designated “K2”, Sigma Chemical Co., St. Louis, Mo.) of the same size and from the same lot.

A murine monoclonal antibody to a conserved human melanoma domain, ganglioside 2, (IgG3, US Biologicals, Swampscott, Mass.) was covalently attached to a short polyaspartate anion chain (MW 3,000) via EDC coupling as above to generate a derivatized antibody designated “Gang2Asp”. Additionally, an anionic imaging agent was designed using an oligonucleic acid as a polyanion wherein the sequence was ATGC-J (designated “ATGC-J” henceforth) with “J” representing a covalently attached Texas Red fluorophore, (Sigma Genosys, Woodlands, Tex.). For this experiment, 6.35 micrograms of Gang2Asp was combined with 0.1712 micrograms of ATGC-J and then complexed with 17.5 micrograms of KNR2 in a total volume of 200 microliters of deionized water to attain a final ratio of 5:1:1::KNR2:ATGC-J:Gang2Asp. The mixture was vortexed for 2 minutes. The resulting complexes were applied to hydrated CellTek Human Melanoma slides and control CellTek Cytokeratin Slides (SDL, Des Plaines, Ill.) and incubated for 5 minutes before photographic evaluation of fluorescence distribution versus brightfield distribution of melanoma pigment in the same field. Additional controls without ATGC-J or without Gang2Asp were also employed.

Results:

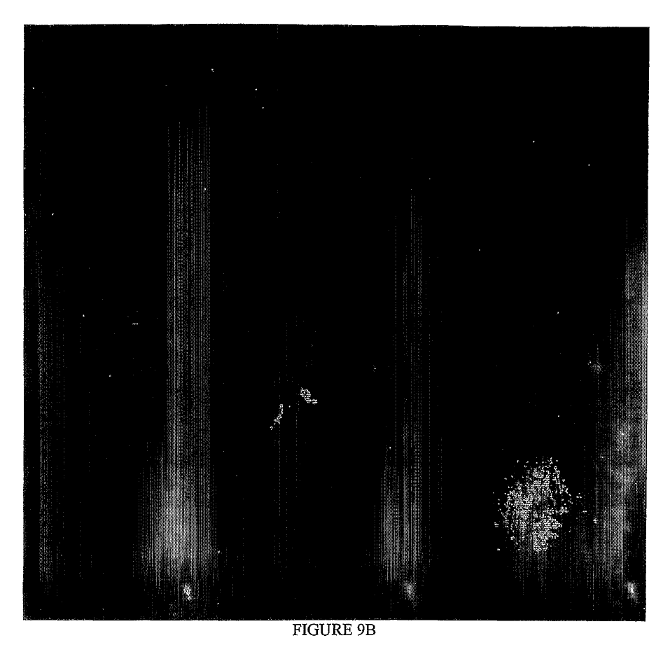

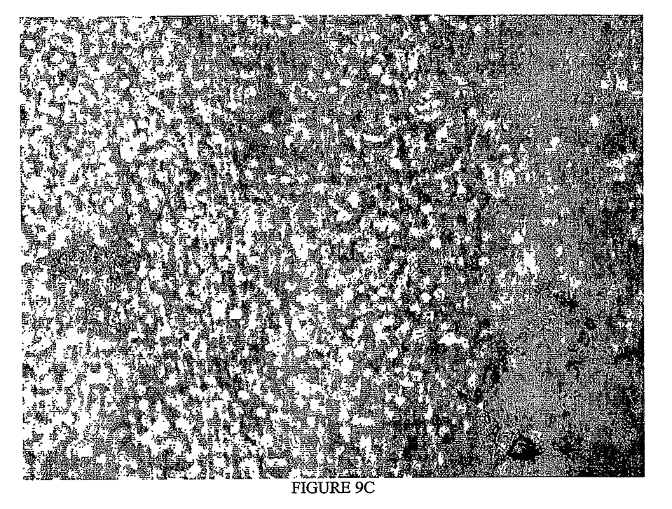

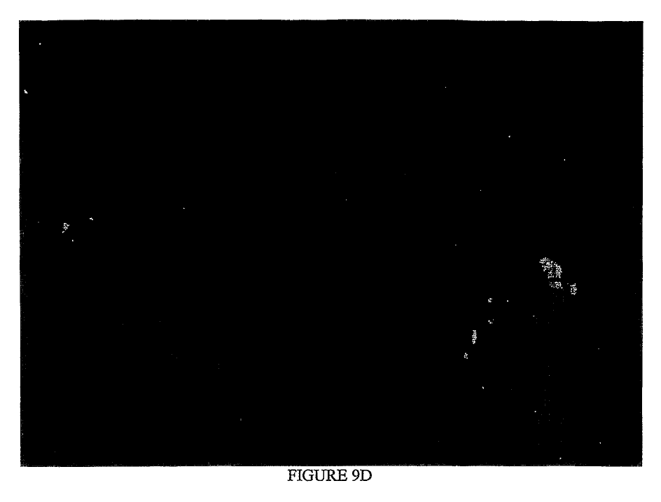

The non-covalent complexes afforded a distribution of the optical imaging agent that followed the tropism of the antibody derivative rather than the distribution of the complexes in the absence of the antibody. More noteworthy, the complexes followed a distribution that matched that of the pigmented melanoma cells, as depicted in FIG. 9.

Conclusions:

This experiment demonstrates the production of a viable complex for transport across skin and visualization of melanoma through optical techniques using a carrier suitable for topical delivery. Such an approach could be employed for example in conjunction with surgical margin-setting or could be employed in routine melanoma surveillance. Similar strategies could readily be employed for topical diagnosis of other skin-related disorders as well, as will be apparent to one skilled in the art. Given the very high sensitivity of optical imaging moieties, significant promise in improved detection of these disorders could be afforded through these non-covalent complexes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-77. (canceled)

78. A method of administering a biologically active protein which is not insulin to a subject, said method comprising topically applying to the skin or epithelium of the subject the protein in conjunction with an effective amount of a positively charged carrier, said positively charged carrier comprising a positively charged backbone having attached positively charged branching groups, wherein the association between the carrier and the biologically active protein is non-covalent.

79. The method according to claim 78 wherein the composition provides greater transdermal delivery of the biologically active protein relative to the agent in the absence of the carrier.

80. The method according to claim 79 in which the biologically active protein has therapeutic activity.

81-83. (canceled)

84. The method according to claim 80 in which the biologically active protein and carrier are administered to the subject in a composition containing both components.

85. The method according to claim 80 in which the biologically active protein and carrier are administered separately to the subject.

86-87. (canceled)

88. The method according to claim 80 in which the composition is a controlled release composition or sustained release composition.

89. (canceled)

90. The method according to claim 80 in which the therapeutic protein is a botulinum toxin.

91. The method according to claim 90 in which the botulinum toxin is selected from botulinum toxin serotypes A, B, C, D, E, F and G.

92. The method according to claim 90 in which the botulinum toxin comprises a botulinum toxin derivative.

93. The method according to claim 90 in which the botulinum toxin comprises a recombinant botulinum toxin.

94. The method according to claim 90 in which the botulinum toxin is administered to provide an aesthetic and/or cosmetic benefit to the subject.

95. The method according to claim 90 in which the botulinum toxin is administered to the subject for prevention or reduction of symptoms associated with muscle spasm or cramping.

96. The method according to claim 90 in which the botulinum toxin and the positively charged carrier are administered topically to a site on the face of the subject.

97. The method according to claim 90 in which the botulinum toxin and the positively charged carrier are administered topically to a site on the subject other than the face.

98-240. (canceled)
